(19) United States
(12) Patent Application Publication
(10) Pub. No.: US 2008/0119530 A1
(43) Pub. Date: May 22, 2008

(54) Method for treatment and prevention of ultraviolet light induced skin pathologies

(76) Inventor: Laura A. Hansen, Bellevue, NE (US)

Correspondence Address: STINSON MORRISON HECKER LLP;ATTN: PATENT GROUP, 1201 WALNUT STREET, SUITE 2800, KANSAS CITY, MO, 64106-2150, US

(21) Appl. No.: 11/906,319

(22) Filed: Oct. 1, 2007

Related U.S. Application Data
(60) Provisional application No. 60/848,108, filed on Sep. 29, 2006.

(51) Int. Cl. A61K 31/428; A61P 17/00; A61P 35/00; A01K 67/027

(52) U.S. Cl. 514/367; 800/18

(57) ABSTRACT

The present invention provides a method for suppression of ultra violet-induced skin pathologies and skin abnormalities, such as abnormal proliferation and mutagenesis, and for inducing apoptosis in cells having Erbb2 or HER2 receptors. The method involves administration of Erbb2/HER2 inhibitors, either before or after exposure to UV.

### CROSS-REFERENCE TO RELATED APPLICATIONS

This application claims the benefit of U.S. Provisional Application No. 60/848,108 filed Sep. 29, 2006 the entirety of which is hereby incorporated herein by reference.

### STATEMENT REGARDING FEDERALLY SPONSORED RESEARCH OR DEVELOPMENT

This invention was supported at least in part by a grant from the National Institutes of Health Grant No. P20 RR018759 and No. P20 RR017717-01, conducted in a facility constructed with support from a Research Facilities Improvement Program Grant Number 1 C06 RR7417-01. The United States Government has certain rights in this invention.

### BACKGROUND OF THE INVENTION

The publications and other materials used herein to illuminate the background of the invention, and in particular cases, to provide additional details respecting the practice, are incorporated in full herein by reference, and for convenience are referenced in the following text by number and are listed numerically in the appended list of references.

UV exposure, like many chemotherapeutics, induces DNA damage and mutations (37). UV-induced DNA damage triggers cell cycle arrest by activating the ATR (Ataxia Telangiectasia mutated Rad3-related) to allow time for repair. The serine/threonine kinase ATR is rapidly phosphorylated and activated following DNA damage. ATR, in turn, phosphorylates and activates Chk1, and to a lesser extent Chk2, kinases that phosphorylate the cell cycle regulator Cdc25a (15; 29). Phosphorylation by Chk1/2 inactivates Cdc25a and targets it for rapid, ubiquitin-directed degradation (15; 34). The Cdc25a phosphatase activates CDK2 by removal of inhibitory phosphate groups at CDK2-Tyr¹⁵ and -Thr¹⁴. Loss of Cdc25a activity following ATR activation thus blocks activation of cyclin/CDK complexes, resulting in S-phase arrest. Cell cycle arrest allows time for the repair of DNA damage and reduces mutagenesis. If cell cycle arrest and DNA repair mechanisms are inadequate, cells acquire mutations that contribute to carcinogenesis.

UV irradiation also activates signaling pathways resembling the response to growth factors that is known as the UV response (21; 54; 55). Interestingly, HER2 (Erbb2 in the mouse) is rapidly activated following UV exposure of skin or cultured keratinocytes through an indirect mechanism involving the inactivation of tyrosine phosphatases, which would otherwise inactivate the receptor. This UV-induced block of phosphatase activity results from UV-generated reactive oxygen species reacting with the cysteine residues conserved at the active site of phosphatases (10; 23; 31; 45).

HER2 is a receptor tyrosine kinase that activates numerous signal transduction pathways regulating cell proliferation and cell death. The Her2 proto-oncogene is activated by amplification, overexpression, or mutation in many kinds of cancer; including mammary, lung, and skin cancer. HER2 overexpression is associated with resistance to chemotherapy and a poor prognosis in mammary and other cancers (49; 65). The mechanisms responsible for chemotherapeutic resistance in HER2-overexpressing cancers were previously unknown, but are of great significance clinically.

In animal models, transgenic overexpression of Erbb2 in the skin results in epidermal and follicular hyperplasia and spontaneous tumor formation (6; 62; 63), demonstrating that increased Erbb2 activation can increase skin carcinogenesis. However, HER2 overexpression is detected in only a subset of nonmelanoma skin cancers associated with more aggressive disease (27; 35).

We and others have shown that EGFR regulates the response of the skin to UV (13). Chemoprevention of UV light-induced skin tumorigenesis by inhibition of the epidermal growth factor receptor. Cancer Res. 65, 3958-3965). UV-induced EGFR activation leads to keratinocyte proliferation, epidermal hyperplasia, suppression of apoptosis and suppression of p53 and p21 expression (14), presumably through the activation of c-Jun NH₂-terminal kinase (JNK), p38 kinase, extracellular signal-regulated kinase (ERK), and phosphatidylinositol 3-kinase (P13K). Reported herein, abrogation of EGFR activity also suppresses UV-induced skin tumorigenesis through the suppression of proliferation, increased apoptotic cell death, and decreased epidermal hyperplasia. While transgenic overexpression of Erbb2 in the skin results in epidermal and follicular hyperplasia and spontaneous papilloma formation (6; 62; 63), a role for Erbb2 in UV-induced skin cancer has not been previously reported.

### BRIEF SUMMARY OF THE INVENTION

The research reported herein reveals a mechanism explaining the resistance of HER2-overexpressing cancers to chemotherapy, documents a novel connection between HER2 and a DNA damage checkpoint, and demonstrates a novel and effective therapy for the treatment or prevention of skin cancer. The epidermal growth factor receptor (EGFR) family includes EGFR (Erbb1), Erbb2, Erbb3, and Erbb4 in the mouse. These receptors are known as EGFR (HER 1), HER2 (Erbb2/neu), HER3 and HER4, respectively in humans. (110) In general the current invention relates to inhibiting any UV-induced skin pathologies in which Erbb2 activity contributes to the pathological condition. It has been discovered that inhibition or knockdown of Erbb2 activity prior to UV irradiation suppresses cell proliferation, cell survival and inflammation after UV, as indicated by decreased mast cell infiltration, edema, and cytokine expression, and skin tumorigenesis, as indicated by both fewer and smaller papillomas and tumors. Specifically, the gene profiling and cell biology experiments reported herein predict a role for normal physiological levels of HER2/Erbb2 in the modulation of the cell cycle after UV irradiation (32). Using a mouse skin model of carcinogenesis, it has been discovered that HER2/Erbb2 suppresses Chk1/2 activation, Cdc25a phosphorylation, Cdc25a degradation and S-phase arrest, thus limiting DNA repair and increasing carcinogenesis. The discovery of many novel genes regulated by Erbb2 reported herein demonstrate the importance of Erbb2 in the response of skin to UV. Erbb2 is necessary for the UV-induced expression of numerous pro-inflammatory genes that are regulated by the transcription factors NF?B and Comp1, including Interleukin-1β (IIIb), Prostaglandin-endoperoxidase synthase 2 (Ptgs2/Cyclooxygenase-2), and multiple chemokines. These results support HER2 as an appropriate target for the treatment and/or prevention of skin cancer. In addition, it has been discovered that inhibition of Erbb2 prior to 5-fluorouracil (5-FU) treatment, a chemotherapeutic that activates the ATR checkpoint, augmented S-phase arrest in keratinocytes. Because many chemotherapeutic agents like 5-fluorouracil activate the ATR-Chk1 cell cycle checkpoint, the results reported herein further reveal a mechanism for the documented resistance of HER2-overexpressing cancer to DNA-damaging chemotherapeutics. The discoveries reported herein, make possible a method for suppressing UV-induced pathologies such as inflammation and skin cancer through pretreatment with a therapeutically effective amount of a compound that inhibits or suppresses Erbb2 activity. The administration of these inhibitors depends on various parameters known to those skilled in the healing arts. The method of the present invention can be used to prevent or inhibit the etiology of UV-induced skin pathologies, such as sunburn, photoaging and skin cancer, as well as in suppressing malignant progression of skin cancers. The method is promising for organ transplant patients with compromised immune systems who often develop aggressive skin cancers, however the present invention can likewise be used to suppress UV-induced skin pathologies and the adverse effects of UV exposure in the general population.

Additional aspects of the invention, together with the advantages and novel features appurtenant thereto, will be set forth in part in the description which follows, and in part will become apparent to those skilled in the art upon examination of the following, or may be learned from the practice of the invention. The objects and advantages of the invention may be realized and attained by means of the instrumentalities and combinations particularly pointed out in the appended claims.

### BRIEF DESCRIPTION OF THE DRAWINGS

FIG. 1A-B shows that Erbb2 regulates cell cycle progression following UV exposure by suppressing checkpoint activation. A) In normal cells, UV-induced Erbb2 activity signals through the PI3K/Akt pathway which decreases the phosphorylation and subsequent degradation of Cdc25a by Chk1. B) Upon abrogation of Erbb2 activity, increased ATR-pathway activity leads to increased degradation of Cdc25a and increased subsequent S-phase arrest.

FIG. 2 shows primary keratinocytes were pre-treated with the vehicle DMSO or AG825 2 h prior to UV- or sham-irradiation. Keratinocytes were harvested at the indicated times after irradiation and immunoblotted as indicated (B) or analyzed for DNA content (A). *Significantly different compared to appropriate vehicle treated control where p=0.05.

FIG. 3A-C show decreased Cdc25a upon abrogation of Erbb2 signaling increases S-phase arrest. A-B) Cdc25a immunoblotting after abrogation of Erbb2 and UV irradiation. Keratinocytes were treated with AG825 or DMSO 2 h before UV (A) or were transfected with Erbb2 siRNA or negative control siRNA two days before UV irradiation (B). Keratinocytes were exposed to 600 J/m² UV, cell lysates collected at various times after UV, and samples immunoblotted as indicated. C) Ectopic expression of Cdc25a repairs the cell cycle defect in keratinocytes lacking Erbb2 activity. Keratinocytes were transfected I d prior to 600 J/m² UV exposure with plasmids containing Cdc25a and GFP or with GFP alone. Twenty-four hours after UV or sham-irradiation, DNA content was determined using propidium fluorescence in cells run through a flow cytometer. N=3. Experiment is representative of three performed. *Significantly different using a Bonferroni posttest on a two-way ANOVA where p=0.01

FIG. 4A-F show that Erbb2 blocks Chk1 activation by ATR through a PI3K/Akt dependent mechanism after UV irradiation. Keratinocytes in culture were treated as indicated and sham-irradiated or exposed to 600 J/m² UV. A) ATR/M activity was determined by immunoblotting for ATR/M substrate phosphorylation using an antibody specific for the phosphorylated consensus ATR/M substrate sequence. N=6. B) Immunoblotting for Chk1 and Chk2 activation upon abrogation of Erbb2 and UV exposure in vehicle or AG825 treated or siRNA transfected keratinocyte cell lysates. C) Akt activation, measured by AKT phosphorylation, is dependent on Erbb2 activation after UV irradiation. D) Inhibition of P13K or Akt causes S-phase arrest after UV irradiation. N=4 experiments. E) Immunoblotting for inhibitory phosphorylation of Chk1 on Ser²⁸⁰ upon abrogation of Erbb2 activity and UV irradiation. F) inhibition of Erbb2 increases S-phase arrest induced by chemotherapeutic 5-FU. *, **,***) Significantly different when compared to the vehicle-treated and UV-irradiated control, the vehicle-treated control, or the 5-FU treated group, respectively, where=p 0.05.

FIG. 5 shows genetic ablation of Erbb2, but not Egfr, results in S-phase arrest. Keratinocytes were exposed to 600 J/m2 UV or sham-irradiated and harvested 24 h later. *Significantly different when compared to the corresponding wild type control or sham-irradiated sample, where p<0.05.

FIG. 6 shows that inhibition of Erbb2 and EGFR suppress UV-induced carcinogenesis. Mice were topically treated with the EGFR inhibitor AG1478 or the Erbb2 inhibitor AG825 2 h prior to UV irradiation. *,**Indicate significant difference from vehicle-treated control and single inhibitors, respectively, where p=0.05 in two-way ANOVA.

FIG. 7 shows that the Erbb2 inhibitor AG825 (left) and EGFR inhibitor AG1478 (right) have distinct cell cycle effects. Skin sections from inhibitor or vehicle treated and UV-exposed (16 hours after for AG1478 treatment; 24 hours after for AG825 treatment) mice were analyzed using flow cytometry. *Significantly different from corresponding vehicle-treated control, p=0.05.

FIG. 8 shows that abrogation of Erbb2 (left) but not EGFR (right) reduces Cdc25a. Control=Erbb2^{flfl}/Cre; Mutant=Erbb2^{flfl}/Cre⁺. Multiple bands are due to ubiquitin conjugation of Cdc25a.

FIG. 9 shows that genetic ablation of Erbb2 reduces UV-induced p53-positive foci.

FIG. 10 shows that HER2-targeted siRNA reduces HER2 in squamous carcinoma cell line SCC12. HER2=HER2-targeted siRNA; (NC)=negative control siRNA; Sham=sham-transfected.

FIG. 11 shows that HER2 expression by carcinoma cells increases angiogenesis. Endothelial cells were cultured with carcinoma cell extracellular matrix or carcinoma conditioned medium.

### DETAILED DESCRIPTION OF PREFERRED EMBODIMENT

Non-melanoma skin cancer, caused primarily by ultraviolet (UV) irradiation, accounts for half of all cancer in the United States. UV exposure also rapidly activates the growth regulatory proto-oncogene Erbb2 (HER2/neu), leading us to hypothesize that Erbb2 activation may deregulate cell cycle control and contribute to UV-induced skin carcinogenesis. The results reported herein show that inhibition of Erbb2 reduced UV-induced skin tumorigenesis by triggering S-phase arrest. It has been discovered that UV-induced Erbb2 activation permits S-phase progression by suppressing the activation of Chk1 and consequent Cdc25a degradation through a phosphatidyl-inositol-3-kinase/Akt-dependent mechanism. Blockade of Erbb2 signaling similarly suppresses S-phase arrest following treatment with the DNA-damaging chemotherapeutic and ATR-activator, such as 5-fluorouracil or camptothecin. Resistance to chemotherapy and poor prognosis are associated with HER2 overexpression in many human cancers. The research reported herein reveals a mechanism explaining the resistance of HER2-overexpressing cancers to chemotherapy, documents a novel connection between HER2 and a DNA damage checkpoint, and demonstrates a novel and effective therapy for the treatment or prevention of skin cancer.

The present invention provides a novel method of suppressing ultraviolet light (UV) induced pathologies, such as skin cancer, based upon pharmacological abrogation of HER2/Erbb2 activity. The abrogation is accomplished by administration of a therapeutically effective amount of a compound that inhibits HER2/Erbb2 activity prior to UV irradiation.

The present invention also provides a method for decreasing skin hyperplasia which is caused, at least partially, by UV irradiation, which comprises administering a therapeutically effective amount of a compound that inhibits HER2/Erbb2 activity.

The present invention further provides a method for suppressing inflammation which is caused, at least partially, by UV irradiation, which comprises administering a therapeutically effective amount of a compound that inhibits HER2/Erbb2 activity prior to UV irradiation.

The present invention also provides a method for suppressing mast cell accumulation which is caused, at least partially, by UV irradiation, which comprises administering a therapeutically effective amount of a compound that inhibits HER2/Erbb2 activity.

The present invention also provides a method for suppressing mutagenesis and the progression of benign precursor lesions to malignant skin tumors which is caused, at least partially, by UV irradiation, which compress administering a therapeutically effective amount of a compound that inhibits HER2/Erbb2 activity.

The present invention also provides a method for suppressing angiogenesis during progression of benign precursor lesions to malignant skin tumors which is caused, at least partially, by UV irradiation, which compress administering a therapeutically effective amount of a compound that inhibits HER2/Erbb2 activity.

Definitions. The present invention employs the following definitions:

“Therapeutically effective amount” is an amount sufficient to suppress and/or inhibit the indicated activity, function or expression.

“Compound(s) that inhibit HER2/Erbb2 activity” and “compound(s) that suppress Erbb2/HER2 activity” refer to compound(s) that act directly or indirectly, and include compounds that result in reduced HER2/Erbb2 activity through an effect on one or more other epidermal growth factor receptor members.

Chronic exposure to UV is the main etiological factor contributing to nonmelanoma skin cancer. The discoveries reported herein demonstrate that the UV-induced activation of Erbb2 (human HER2) promotes UV-induced skin tumorigenesis. Erbb2 activation by UV occurs rapidly and subsides within 40 minutes of exposure to UV. In addition, the inhibition of Erbb2 by AG825 lasts less than 24 h. With such a short timeframe of receptor activation and inhibition, it is remarkable that 12 weeks later, both tumor multiplicity and tumor volume are decreased by more than half. These results demonstrate a powerful effect of UV-induced Erbb2 activation on the promotion of skin tumors. The discoveries reported herein make possible a method for the use of Erbb2 inhibitors as a strategy for decreasing the incidence of nonmelanoma skin cancer, the most prevalent form of cancer in the United States. Low incidence of skin toxicity has been shown with current Erbb2-targeted therapies, such as trastuzumab, pertuzumab, and CP-724714.

The mechanism by which inhibition of Erbb2 blocks skin tumor development was investigated initially by examining both cell division and cell death in Erbb2 inhibitor-treated and UV-exposed skin. Erbb2 inhibition using AG825 or Erbb2-specific siRNA in vitro consistently results in a small increase in apoptosis after UV, which varies in its extent depending upon the assay. Part of the oncogenic potential of Erbb2 is its activation of the PI3K/AKT survival pathway (39; 59). Breast cancer cell lines overexpressing Erbb2 were found to have increased AKT2 expression and resistance to UV-induced apoptosis, which was reversed by inhibiting PI3K (3). However, TUNEL analysis demonstrated no suppression of UV-induced apoptosis by Erbb2 in the skin.

Epidermal hyperplasia is strongly correlated with increased proliferation during skin tumor promotion. Consistent with the results reported, previous studies have shown that UV-induced epidermal hyperplasia is maximal within the first 2 d after the first or second UV exposure (13). Inhibition of Erbb2 suppressed proliferation and the development of epidermal hyperplasia due to increased S-phase arrest. It has been discovered that UV-induced Erbb2 activation allows for DNA synthesis after UV exposure. In the absence of Erbb2, DNA synthesis largely stops by 12 h after UV and keratinocytes accumulate in S-phase several hours later. Irradiation of keratinocytes has previously been reported to cause S-phase delay or arrest (30; 43). However, multiple mechanisms were proposed by others to account for UV-induced cell cycle arrest. UV-induced DNA damage causes p53 activation, increased p21 activation and subsequent S-phase delay or arrest in keratinocytes (42). In contrast, abrogation of Erbb2 did not increase p21 expression in the experiments reported herein. Based on these data, it was hypothesized that Erbb2 dampens activation of the ATR DNA damage cell cycle checkpoint, known to cause S-phase arrest following UV exposure. These investigations demonstrated that Erbb2 does not affect the activation of ATR itself but rather the Chk1/2 kinases downstream from ATR. Erbb2 activation of PI3K/Akt signaling causes inhibitory phosphorylation of Chk1 on Ser²⁸⁰, substantially blocking Chk1 activation by ATR and reducing Cdc25a degradation following UV irradiation. The mechanism by which Erbb2 regulates Chk2 activation may involve Akt activation as well. A bioinformatics analysis revealed two regions of Chk2 with sequences very similar to the Akt consensus sequence that were centered on Ser¹²⁴ and Ser¹⁴⁴, consistent with the hypothesis that Akt can phosphorylate Chk2.

It is known that activated Chk1/2 phosphorylates the Cdc25 family of phosphatases (Chen et al. 2003) (Zhao et al., 2002), inactivating them (Uto et al., 2004) and targeting them for rapid degradation (34; 61). The Cdc25 family consists of Cdc25a, Cdc25b, and Cdc25c which dephosphorylate and activate CDK2, Cdc2 and Cdc3, respectively. Among these family members, only Cdc25a was significantly reduced upon loss of Erbb2 signaling. Cdc25a activates CDK2, which complexes with either cyclin E or cyclin A, and is involved in S-phase progression (5; 46; 47). S-phase arrest following Chk1 activation and Cdc25a degradation is also regulated by the PI3K pathway. Inhibition of PI3K results in the activation of Chk1 (48) and increased S-phase arrest in leukemia cells (7). In a hypoxia-induced S-phase arrest model, cells with constitutively active Akt did not succumb to S-phase arrest (8). In PTEN^{-/-} cells, which exhibit a defective cell cycle checkpoint response to ionizing radiation, elevated Akt activity led to inhibitory Chk1 phosphorylation on Ser²⁸⁰ and increased hypophosphorylated Cdc25a (44). These data reveal a connection between PI3K signaling and inhibition of the ATR DNA damage checkpoint. The results presented herein further document that Erbb2 activation upon UV irradiation modulates the ATR pathway by activating PI3K and Akt.

Alternative mechanisms of modulation of the ATR DNA damage response pathway have been demonstrated. Inactivation of Cdc25a can occur through activation of a member of the p38 MAPK signaling pathway, which is also activated by Erbb2, but this regulation of Cdc25a remains controversial (24). The present data demonstrates that Erbb2 inhibition decreases p38 kinase activity after sham irradiation and 6 h after UV irradiation, times at which Cdc25a is also decreased, which does not support this mechanism for Erbb2's effects on the cell cycle. Surprisingly, Erbb2 inhibition causes p38 kinase activity to increase 12 and 24 h after UV concomitant with decreased Cdc25a expression, potentially implicating this pathway as a delayed mechanism to cause S-phase arrest. In addition, siRNA knockdown of Erbb2 did not have this effect on p38 kinase. Other mechanisms for the inactivation of Cdc25a, including sequestration in the cytoplasm and phosphorylation by p38 kinase following UV exposure or osmotic shock, have been reported (25). The use of AG825 caused S-phase arrest after sham UV which may be due to the observed Cdc25a degradation but not through MAPK signaling mechanisms due to the observed decrease in p38 kinase, ERK1/2 and JNK1/2 activity.

Inhibition of Erbb2 by herceptin has also been shown to decrease PI3K activity and increase p27 expression, resulting in inhibition of CDK2 (64). The results reported herein demonstrate that Erbb2 signals through this pathway in addition to PI3K, thus Erbb2 may modulate S-phase progression through a p27-dependent mechanism as well. Independent of UV irradiation, PI3K signaling has been linked to Erbb2 and cell cycle progression. Overexpression of Erbb2 activates Akt in breast cancer cells to allow S-phase progression (53). However, a connection between Erbb2 and a DNA damage checkpoint has not been previously established. The role of DNA damage-induced cell cycle inhibition after UV irradiation of the skin is generally thought to allow for DNA repair and subsequent cell cycle reentry. However, recent evidence suggests that keratinocytes arrested in S-phase move away from the basement membrane into more differentiated layers and are then lost from the surface of the skin (51). This suggests that S-phase arrest in UV-exposed keratinocytes may permanently block cell cycle reentry, implicating an alternative mechanism for their removal instead of through apoptosis. However, if this mechanism for the elimination of damaged cells from the skin is correct, UV-induced Erbb2 activity would override the S-phase arrest and subsequent removal of the damaged cell. Since Erbb2 is overexpressed in some nonmelanoma skin cancer, the role of UV-induced Erbb2 activity in suppressing ATR-mediated S-phase arrest may be a mechanism by which cells with DNA damage avoid apoptosis. These results reported herein demonstrate that Erbb2 inhibition decreases proliferation after UV due to cessation of DNA synthesis rather than an increase in cell death. These results contrast with previous publications using tumor cells overexpressing Erbb2 in which a block in Erbb2 primarily leads to apoptosis (60). Thus, the biological functions of Erbb2 when expressed at normal physiological levels may differ from its effects when overexpressed. Most previous research has focused on Erbb2 signaling in tumor cell lines.

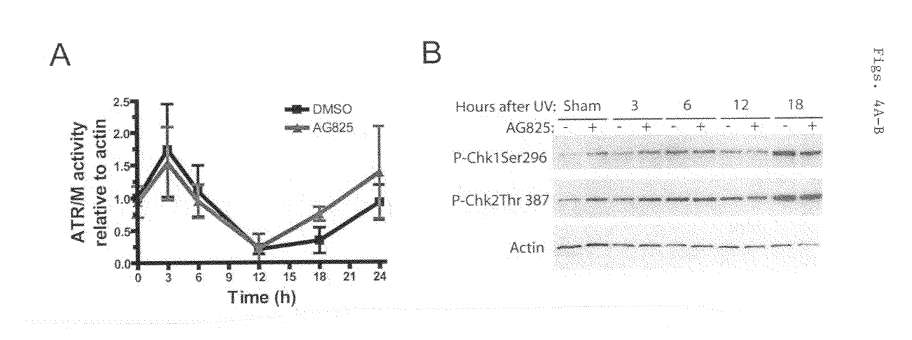

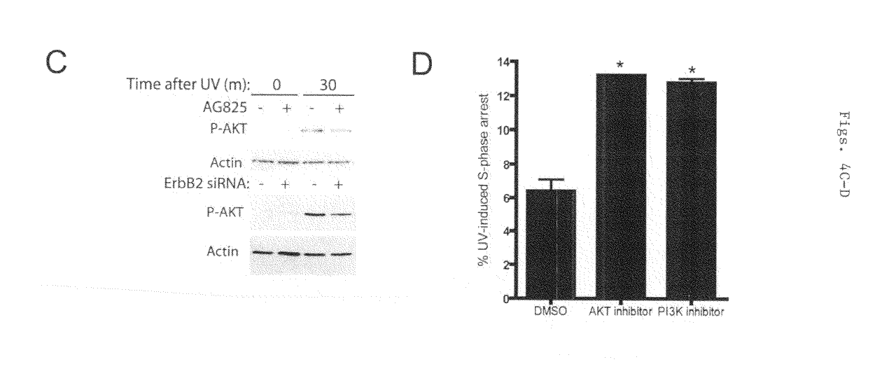

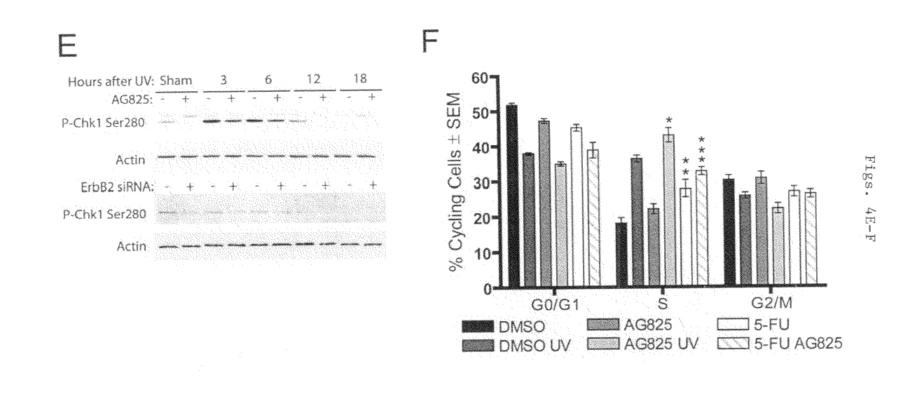

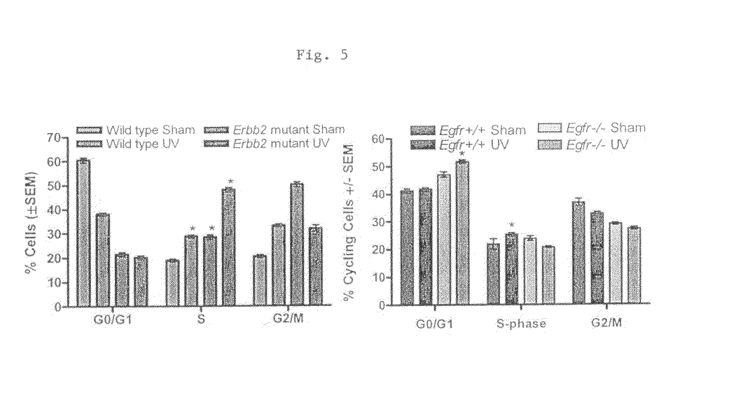

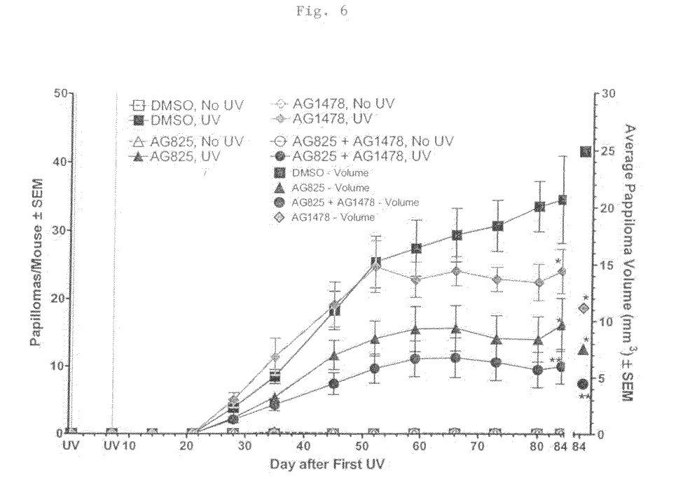

Surprisingly given that Erbb2 heterodimerizes with EGFR, we found that EGFR signaling has effects quite distinct from Erbb2. We and others have shown that EGFR regulates the response of the skin to UV (13; 19; 57; 58). UV-induced EGFR activation leads to keratinocyte proliferation, epidermal hyperplasia, suppression of apoptosis and suppression of p53 and p21 expression (14), presumably through the activation of c-Jun NH₂-terminal kinase (JNK), p38 kinase, extracellular signal-regulated kinase (ERK), and phosphatidylinositol 3-kinase (P13K). Abrogation of EGFR activity also suppressed UV-induced skin tumorigenesis through a suppression of proliferation, increased apoptotic cell death, and decreased epidermal hyperplasia (13). However, EGFR does not promote S-phase progression or maintain Cdc25a following UV irradiation (FIGS. 4 and 7). Suppression of Chk1 activation by EGFR has recently been reported, although this mechanism triggers G₂/M-phase arrest (41). The mechanisms for signaling specificity downstream from EGFR and Erbb2 resulting in such distinct effects on the cell cycle remain unexplained.

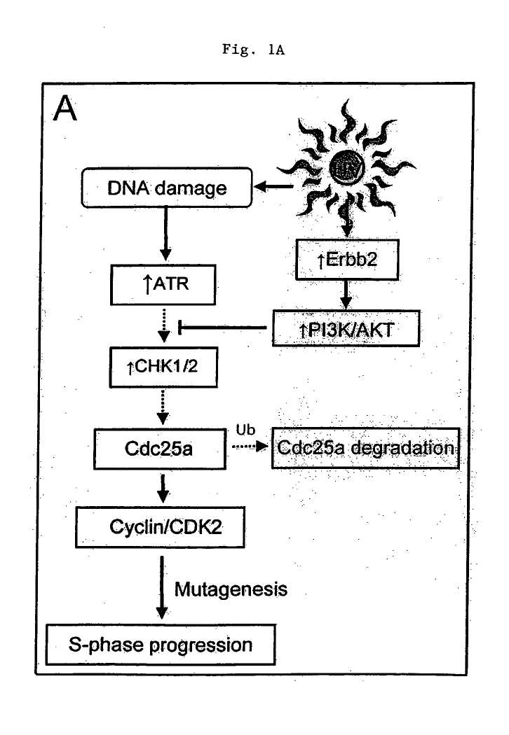

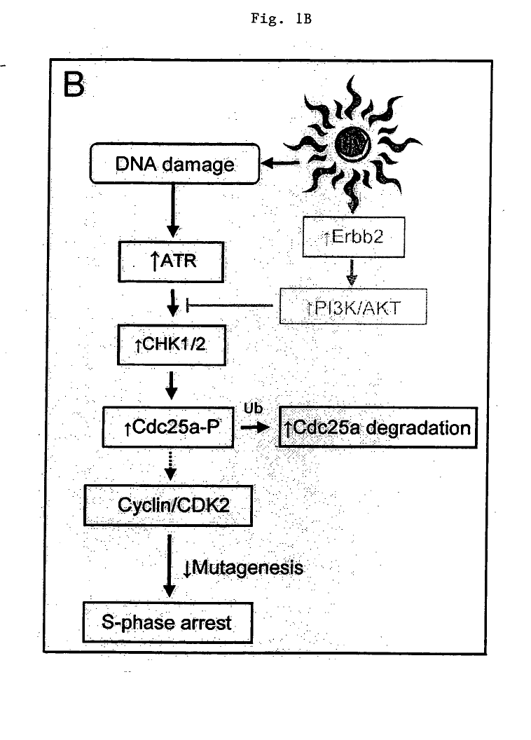

While not wanting to be bound by any single explanation one model that explains the results reported herein is that Erbb2 works upstream of a DNA damage sensitive cell cycle checkpoint, revealing a novel crosstalk between Erbb2 and a DNA damage response pathway. Erbb2 activation following UV irradiation activates PI3K/Akt signaling, resulting in inhibitory phosphorylation of Chk1 on Ser²⁸⁰ and blocking activation of Chk1 and Chk2 by ATR (FIG. 1A). Without strong Chk1/2 activation, Cdc25a phosphorylation and degradation is reduced and keratinocytes do not completely cease synthesizing DNA. Loss of Erbb2 in contrast, allows for full activation of Chk1/2, Cdc25a degradation, and increased S-phase arrest (FIG. 1B). The ATR DNA-damage response mechanism has been implicated in early tumorigenesis (4) and is activated by chemotherapeutic agents such as 5-FU and camptothecin (2, 9). Additionally, amplification of Erbb2 is associated with resistance to chemotherapy in human adenocarcinomas (36). The results reported herein demonstrate that Erbb2 suppresses cell cycle arrest following 5-FU treatment, providing a mechanistic explanation of these clinical data. Thus, Erbb2 may be an important target that increases the response to ATR/M-activating chemotherapeutics like 5-FU.

Previous studies designed to assess the global effects of UV on the skin have used microarray analysis performed on primary keratinocytes and epidermis exposed to UV (19; 24; 49; 64; 77; 86). Consistent with our results, these studies have determined that UV modulates hundreds of genes involved in diverse processes such as proliferation, apoptosis, inflammation, cell adhesion and migration. Using global expression analysis by Affymetrix microarray, we have characterized the transcriptome in the skin modulated by the transient activation of Erbb2 after UV exposure and identified many novel genes regulated by Erbb2. In addition, our analysis revealed that Erbb2 modulates proliferation and inflammation with a lesser but statistically significant effect on apoptosis following UV irradiation.

Stringent criteria were used in this analysis. The inherent variability among in vivo samples led to the statistical determination of the need for microarray experiments to be performed in at least triplicate (50). We used biological replicates in quadruplicate for an additional margin of confidence. Microarray analysis is complex and no two papers seem to use identical criteria. Therefore, multiple sets of criteria were used both to determine probe intensities and to identify genes that were significantly changed. Only genes that met all these criteria were reported herein. Real-time PCR validated the microarray data while scatter plots and clustering revealed global trends in gene expression. Selected biological pathways were validated experimentally, which further confirmed the in silico analysis.

Since receptor tyrosine kinases like Erbb2 are generally thought to activate signal transduction cascades leading to transcription factor activation, gene expression was expected, for the most part, to be decreased upon Erbb2 inhibition. Contrary to what was expected, gene expression was more often increased than decreased after its inhibition. Thus, UV-induced activation of Erbb2 did more to suppress or inhibit global gene expression than to increase transcription. There were notable exceptions to this trend, however, such as the suppression of expression of numerous immune response genes upon inhibition of Erbb2 and UV exposure. The significance and mechanisms of suppression of gene expression by Erbb2 remain to be determined. Interestingly, microarray analysis of Egfr null and wild type control skin revealed a similar pattern of suppression of gene expression by the receptor (unpublished data). Published studies comparing gene expression in cells with normal and increased Erbb2 expression have found varied responses in global gene expression. Over-expression of Erbb2 led to either more frequently increased (55) or decreased (101) gene expression in mammary luminal epithelial cells. Gene expression was more frequently increased in erbB2-overexpressing breast cancer cell lines, but in primary breast tumors, gene expression was more frequently decreased when Erbb2 is overexpressed (102). Therefore, no consensus about the global effects of Erbb2 on gene expression has yet emerged from these studies.

A novel role for Erbb2 in the induction of inflammation after UV was identified. We found that transient Erbb2 activation augments UV-induced inflammation and regulates the expression of a myriad of effectors of this response. Many of these effectors are known to be regulated by NFκB, consistent with the PAINT analysis indicating NFκB as an Erbb2-regulated transcription factor. Two NFκB-regulated candidates (reviewed in 6) for Erbb2-induced inflammation after UV exposure are Il1 b and Ptgs2, both potent mediators of inflammation. The UV-induced expression of both Il1 b and Ptgs2 was largely dependent upon Erbb2 activation. Ptgs2 has also been shown to be regulated by Erbb2 in colorectal cells (94). A potential mechanism for Erbb2 activation of NF?B is the PI3K pathway, as documented in mammary epithelial cells (reviewed in 109). Erbb2 may also suppress the inhibition of NF?B by Pawr (27). Our data also link Erbb2's proinflammatory effects to the little investigated Comp1, since 7 Erbb2-regulated, pro-inflammatory genes had Comp1 binding sites. These genes included Ccl4, which is important in acute inflammatory responses and also plays a role in wound reepithelialization and collagen synthesis (54). The mechanisms through which Erbb2 alters NF?B and Comp1 signaling to increase inflammation require further investigation.

Other genes whose expression was modulated by Erbb2 that may be involved in the inflammatory response include Ptprc. The expression of Ptprc, which is involved in the migration of inflammatory cells after UV exposure (4; 76; 82), was decreased upon inhibition of Erbb2. Many UV-regulated cytokines that activate or chemoattract inflammatory cells such as Ccl12, Ccl11 and Cxcl2 were regulated by Erbb2. Ccl11, in particular, induces the migration of Cd53-positive mast cells to the skin (73). Cd53 is upregulated to protect against oxidative stress and UVB. While mast cells are best known for their role in allergic reactions, they are also important in the response to stress, and are induced in the skin after UV exposure (reviewed in 60). Thus, Erbb2 may induce mast cell migration by increasing Ccl11 expression. Changes in the expression of inflammation-related genes may also occur through an indirect mechanism involving cellular changes that alter the response of the stroma or tissue infiltrate. Analysis of the expression of cytokines and other inflammatory gene products in skin and cultured keratinocytes lacking Erbb2 activity is underway to distinguish between these possibilities.

The skin contains many defenses for protection from environmental insults and oxidative stress. The generation of ROS by UV is met by a quick response of increasing both detoxifying enzymes and inflammatory mediators to maintain normal homeostasis. The response is partly due to the activation of redox-sensitive transcription factors (1). However, UV-generated ROS also result in the activation of Erbb2, which as our results demonstrate, increases inflammation. These data indicate that a portion of the inflammation caused by UV-induced ROS occurs indirectly as a result of Erbb2 activation.

Inhibition of Erbb2 altered the expression of many proliferation-associated genes after UV as well. The sharp decline in cyclin expression 6 h after UV exposure was consistent with cell cycle arrest after UV. The skin, however, must maintain its integrity and replenish lost cells; this is illustrated by the rebound of cyclin gene expression and BrDU incorporation 24 h after UV exposure. Tk1 expression, which is involved in the synthesis of DNA and is a marker of cell proliferation (98), followed the same pattern of gene expression demonstrated by the cyclins in our experiments. Inhibition of Erbb2, however, altered the pattern of expression of these genes such that their decrease in expression at 6 h after UV was not as great but neither was the recovery of expression at 24 h. In some cases, the expression of the genes continued to decrease at 24 h after UV exposure. These data suggest that Erbb2 deregulates cell cycle arrest after UV exposure as well as the recovery of proliferation at later times. Indeed, our data showed that Erbb2 is necessary for keratinocyte proliferation after UV exposure. The mechanisms by which Erbb2 modulates the expression of cell cycle genes and cell cycle progression warrant further investigation.

While not as dramatic of an effect as on inflammation or proliferation, abrogation of Erbb2 also reduced survival after UV and regulated apoptosis-associated genes. This may be the result of increased expression of proapoptotic genes like Pawr (26) and Sox4 (36) after UV. Additionally, Pdcd4 expression, which is induced during apoptosis (79) and induced by the Erbb2 antagonist herceptin in breast cancer cells (2), was similarly increased. The role of Erbb2 in promoting cell survival may also occur through increased antiapoptotic gene expression, such as Ghr (18) and Ptgs2, which has been shown to promote survival by decreasing apoptosis after UV exposure to keratinocytes (90). Consistent with these data, cell culture experiments to validate the microarray analysis demonstrated that inhibition of knockdown of Erbb2 increased apoptosis slightly but significantly after UV, implying Erbb2 enhances cell survival after UV. However, the biological significance of an effect of this magnitude remains unclear. Although the number of apoptosis genes with increased expression upon Erbb2 inhibition was similar at 6 and 24 h post-UV, an increase in apoptosis was not detected until 24 h, most likely reflecting the time required for protein synthesis and pro-apoptotic signaling to manifest in DNA cleavage.

Our microarray analysis and cell culture experiments revealed that inhibition of Erbb2 decreased proliferation, increased apoptosis, and suppressed inflammation after UV irradiation, all processes intimately linked to cancer development and progression. Fifty-four genes regulated by Erbb2 were linked to cancer in the literature, many of these specifically associated with skin cancer. For example, Ptgs2 expression is increased in many cancers including skin cancer (87) and Hifla is overexpressed in squamous cell head and neck cancer correlated with aggressive behavior and resistance to chemotherapy (43). Inhibition of Erbb2 reduced the expression of Map3k8, a proto-oncogene that is known to act simultaneously on all known MAPK cascades (14), consistent with the activation of MAPK signaling in head and neck squamous cell carcinoma (68). Erbb2 also activates NF-?B, which is increasingly recognized as important in cancer (reviewed in 40) and is constitutively activated in head and neck squamous cell carcinoma (13). While the link between inflammation and cancer is complex (reviewed in 67), changes in inflammatory gene expression have been demonstrated in basal cell carcinoma (100). Ghr expression, suppressed upon inhibition of Erbb2, is a marker for the progression from actinic keratosis to squamous cell carcinoma (83). Significant correlations have been shown between Mmp9, reduced by the Erbb2 inhibitor, and Erbb2 expression with respect to clinico-pathologic parameters in HNSCC (21) and oral squamous cell carcinomas have higher expression of Mmp9 (37). These data support a multifaceted role for Erbb2 in skin cancer development and progression.

Our analysis revealed that Erbb2 not only activated NF?B, but also Comp1, FoxJ2 and Tall. While not much is known about Comp1 and FoxJ2 specifically, deregulation of Fox family members is involved in carcinogenesis (reviewed in 41) and inflammation (53). Tall has a role in hematopoiesis, migration and angiogenesis (46). Transgenic expression of Tall can cause malignancies (17) and its loss can induce apoptosis (51).

EXPERIMENTAL PROCEDURES: The following preparations and methodologies, or those specifically set forth below in the Examples were utilized.

Animals. Homozygous v-rasHa transgenic mice on an FVB/N background were used in in vivo experiments. To obtain keratinocytes with genetic ablation of Erbb2, loxP sites were inserted flanking exon 2 of Erbb2 such that splicing from exon1 to downstream introns upon Cre recombinase expression creates a frameshift mutation. Mice were maintained in our animal facility and provided with Purina lab chow (Nestle Purina PetCare, St. Louis, Mo.) and water ad libitum. The dorsal skin was clipped one day before treatment using electric clippers (Wahl, Sterling, II) and shaved on the day of treatment with a Remington Microscreen shaver (Madison, N.C.). Four mg AG825 (AG Scientific, San Diego, Calif. and Calbiochem, San Diego; CA) dissolved in 200 μl DMSO, or the vehicle alone, was applied topically to the shaved back of the mice 2 h prior to exposure to 10 kJ/m² UV or sham irradiation. The Ultraviolet-B TL 40W/12 RS bulbs (Philips, Somerset, N.J.) used emitted approximately 30% UVA, 70% UVB and <1% UVC, with a total output of 470 μW/cm², as measured with radiometric photodetector probes (Newport, Irvine, Calif.). For tumor experiments, homozygous Tg.AC mice were treated and exposed twice with an interval of 7 d. Tumor number was counted and tumor volume measured weekly using calipers. Skin-fold thickness was measured using calipers (Mitutoyo, Aurora, Ill.) to lightly pinch the dorsal skin. All animal procedures were performed in accordance with American Association of Laboratory Animal Care guidelines and approved by Creighton University's Institutional Animal Care and Use Committee.

Adult female CD-1 mice were maintained in our animal facility and provided with Purina lab chow (Nestlé Purina PetCare, St. Louis, Mo.) and water ad libitum. The dorsal skin was clipped one day before treatment and shaved with a Remington Microscreen shaver (Wahl, Sterling, II) on the day of treatment. Dimethyl sulfoxide (DMSO) or 4 mg AG825 (AG Scientific, San Diego, Calif.) dissolved in DMSO was applied topically to the shaved backs of the mice 2 hours before exposure to 10 kJ/m² UV or sham irradiation. UVB TL 40W/12 RS bulbs (Philips, Somerset, N.J.) were used that emitted approximately 30% UV-A, 70% UV-B and <1% UV-C, with a total output of 470 μW/cm², as measured with radiometric photodetector probes (Newport, Irvine, Calif.). Skin-fold thickness was measured using calipers to lightly pinch the dorsal skin. All animal procedures were approved by our Institutional Animal Care and Use Committee.

Any suitable inhibitor known in the art that blocks UV-induced activation of Erbb2 may be used in the method of the present invention including, but not limited to, AG825, CP-724714 (Clinical trials gov. Identifier NCT 00102895; (http://www.clinicaltrials.gov/ct/show/NCT00102895?order=2), Trastuzumab herceptin (2-4 mg/kg body weight i.v. (intravenously) given daily, referenced in (Hurley Doliny, Reis, Silva, Gomex-Fernandez, Velez, Pauletti, Powell, Pegram, Slamon Journal of Clinical Oncology 24 (12):1831-1838, 2006), Lapatinib (Lackey K E Lessons from the drug discovery of lapatinib, a dual Erbb1; /2 tyrosine kinase inhibitor. Curr Topics Med. Chem. 2006:6(5)435-60) or one of the numerous HER2 inhibitors in clinical trials (Resistance of HER2-Targeted Therapy in Breast Cancer, Nat. Clin. Pract. Oncol. 2006, 3(5):269-280), for example in late stage cancer trials such as, 572016 (GlaxoSmithKline), Tarceva (Genetech, OSI Pharmaceuticals), or HER2 antagonists, such as APC 8024 (Dendreon Corp.), CI-1033 (Pfizer, NCI), or PX-104.1 (Pharmexa). The inhibitors can be administered by any effective route, including, without limitation, topically, intraperitoneally (13) or orally (Example 5). One skilled in the art will appreciate that suitable routes of administering inhibitors of the method of the present invention to a mammal, in particular a human, are available, and, although more than one route can be used to administer an inhibitor, a particular route can be more immediate or effective than another route. Accordingly, the herein-described routes are merely exemplary and are in no way limiting. The appropriate dose and dose frequency will depend on the route of administration and the treatment indicated, and can be readily determined by a skilled artisan, such as by extrapolation from current treatment protocols. For example, immunocompromised individuals may need to be treated for years to prevent squamous cell carcinoma. It will be recognized that, while any suitable carrier, adjuvant and/or additive known to those of ordinary skill in the art may be employed to administer the compositions of this invention, the type of carrier adjuvant and/or additive will vary depending on the mode of administration. Therefore, any appropriate carrier, adjuvant or additive may be included along with the inhibitor compound in the practice of the present invention, including without limitation, a preservative, organic solvent, stabilizer, emollient moisturizing agent, UVA/UVB filer, dermal-penetrant, lipid, ester, diluents, emulsion, oil, or gel.

Immunoblotting. Flash frozen skin was ground with a mortar and pestle on dry ice, homogenized in lysis buffer containing 10 mM Tris (pH 7.4), 150 mM NaCl, 10% glycerol, 1% Triton X-100, 1 mM EDTA, Complete Protease Inhibitor Cocktail (Roche, Germany), 1 mM Na₃V O₄, 1.5 μM EGTA, and 10 μM NaF. Protein was quantified using the Coomassie Brilliant Blue G-250 protein assay (Bio-Rad, Hercules, Calif.). The evenness of loading and transfer was determined by staining with Ponceau S and by actin immunoblotting. Membranes were immunoblotted with antibodies recognizing actin (Sigma, St. Louis, Mo.), phosphorylated ATR/M substrates (Cell Signaling, Danvers, Mass.), Cdc25a (Santa Cruz Biotechnology, Santa Cruz, Calif.), Cdc25b (Cell Signaling), Cdc25c (Santa Cruz Biotechnology), phospho-Cdc25a Thr-506 (Cell Signaling), phospho-Chk1-Ser-296 (Cell Signaling), phospho-Chk1Ser²⁸⁰ (gift of Ramon Parsons), phospho-Chk1-345 (Santa Cruz), phospho-Chk2-387 (Cell Signaling), p21 (Santa Cruz Biotechnology), p27 (Santa Cruz Biotechnology, Santa Cruz, CA), and phospho-Akt (Cell Signaling, Beverly, Mass.), horseradish peroxidase-conjugated secondary antibodies (Cell Signaling, Beverly, Mass.), and visualized using chemiluminescent reagents (Pierce, Rockford, Ill.).

For some experiments, the epidermis was separated from the skin by the heat shock method before homogenization. Keratinocytes were lysed in the same buffer. Membranes were immunoblotted with antibodies recognizing Erbb2, phospho-Erbb2 (Tyr¹²⁴⁸), EGFR, phospho-EGFR (Tyr⁹⁹²), phospho-EGFR (Tyr¹¹⁷³), phosphotyrosine, and Tall (Calbiochem, San Diego, Calif.). Binding of horseradish peroxidase-conjugated secondary antibodies (Cell Signaling, Beverly, Mass.) was visualized using chemiluminescent reagents (Pierce, Rockford, Ill.) and autoradiography. Densitometry was performed using IDScan software (Scanalytics, Fairfax, Va.).

Microscopy. Hematoxylin and eosin staining was performed on paraffin-embedded sections following standard protocols. Following antigen retrieval, paraffin-embedded sections were incubated with antibodies to keratin 1, keratin 6, filaggrin (all from Covance, Princeton, N.J.), or TNFμ (R&D Systems, Minneapolis, Minn.), Alexa Fluor 488-conjugated secondary antibody (Molecular Probes), and 4′,6-diamidino-2-phenylindole (DAPI; Vector Laboratories, Burlingame, Calif.). Apoptotic cells were identified using terminal deoxynucleotidyl transferase-mediated dUTP nick end labeling (TUNEL; Promega, Madison, Wis.).

Flow cytometry. 60 μM sections from paraffin embedded blocks were dewaxed with xylene, rehydrated and digested in PBS containing 0.5% pepsin (Sigma, St. Louis, Mo.). The nuclei were isolated by filtration and suspended in Vindelov's (56) solution reagent (3.5 mM tris base, pH 7.6, 10 mM NaCl, 10 μg/ml Ribonuclease A, 75 μg/ml PI, 1.0 μl/ml IPEGAL; (71)). At least 10,000 events from each sample were analyzed for flow cytometry on a FACSCalibur flow cytometer (Becton Dickinson, Franklin Lakes, N.J.). Data were acquired in the FL2 channel with a 585/42 bp filter. Fluorescence signals were pulse processed and single cells identified using a FL2W versus FL2A plot. A histogram of FL2A was plotted for single cells and cell cycle distributions determined using ModFit LT 3.1 software (Verity Software House, Topsham, Me.).

Cytokine Profile. Whole skin was obtained from mice 8 hours after being treated topically with AG825 or DMSO and sham irradiated one week apart. Whole skin lysate and cell culture lysate was prepared in 1 X CHAPS lysis buffer (Chemicon, Temecula, Calif.). Protein was quantified as above and sent to CytokineProfiler Testing Service (Upstate, Charlottesville, Va.) for analysis of interleukin 1a (II-1a) concentration using Luminex xMAP technology.

Cell culture. Primary newborn mouse keratinocytes isolated as described previously (20) were grown to 70% confluence; treated with 45 μM AG825 (AG Scientific, San Diego, Calif.), 1 μM 5-FU (Calbiochem), 15 μM LY294002 (Calbiochem, La Jolla, Calif.), or 15 μMμAkt inhibitor IL-6-hydroxymethyl-chiro-inositol-2-20-methyl-3-O-ocadecylcarbomate (Calbiochem) and exposed to 600 J/m² UV or sham irradiated as described previously (32). Keratinocytes were transfected with Erbb2-targeted siRNA (32), Stealth RNAi Negative Control LO GC (Invitrogen, Carlsbad, Calif.), Cy3-conjugated Label IT RNAi Delivery Control (Mirus, Madison, Wis.) or sham transfection with TransIT-siQUEST Transfection Reagent (Mirus, Madison, Wis.). Plasmids with Cdc25a (Hassepass et al. 2003a) and EGFP (enhanced green fluorescent protein) cDNA were cotransfected with Lipofectamine Transfection Reagent (Invitrogen, Carlsbad, Calif.). Transfection efficiency was quantified by determining the proportion of the cells that incorporated the fluorescent Cy3-conjugated siRNA or GFP one day post-transfection. Keratinocytes homozygous for the Erbb2 loxP mutation were infected with Cre recombinase-expressing or empty adenoviral vectors in polybrene (Sigma).

Cell cycle. For in vivo analysis of cell cycle distribution, sections from paraffin embedded blocks were dewaxed with xylene, rehydrated and digested in PBS containing 0.5% pepsin (Sigma, St. Louis, Mo.). The nuclei were isolated by filtration and suspended in Vindelov's solution reagent (3.5 mM tris base, pH 7.6, 10 mM NaCl, 10 4 μg/ml Ribonuclease A, 75 μg/ml PI, 1.01l/ml IPEGAL) (56). For in vitro analysis, keratinocytes were resuspended, fixed in 70% ethanol, resuspended in Vindelov's solution (3.5 mM Tris base, pH 7.6, 10 mM NaCl, 10 μg/ml Ribonuclease A, 75 μg/ml propidium iodide, 1.01l/ml IPEGAL), and analyzed on a FACSCalibur flow cytometer (Becton Dickinson, Franklin Lakes, N.J.). Cell cycle distributions were determined using ModFit LT 3.1 software (Verity Software House, Topsham, Me.). Some cells were treated with 10 μM BrDU (Sigma) prior to harvest, incubation with a FITC-conjugated anti-BrDU antibody (BD Biosciences, San Diego, Calif.), incubation with propidium iodide, and flow cytometric analysis.

Statistical methods. The tumor experiment was analyzed using two-way ANOVA. Statistical significance in other experiments was assessed using two-way ANOVA or Student's t-test with Bonferroni post-test. All experiments, excluding the tumor experiment, were replicated several times and consistent results obtained.

Microarray Analysis. Flash frozen skin was ground to a powder using a mortar and pestle and RNA extracted using a PowerGen 700 tissue homogenizer (Fisher, Hampton, N.H.) in TRIzol reagent (Invitrogen, Carlsbad, Calif.) according to the manufacturer's protocol. Total RNA was further purified using RNeasy Midi Columns according to the manufacturer's protocol (Qiagen, Valencia, Calif.). The quality of the RNA was assessed using an RNA 6000 Pico Assay (Aligent, Palo Alto, Calif.) with a Bioanalyzer (Agilent, Palo Alto, Calif.). Mouse MOE430A gene chips were purchased from Affymetrix (Santa Clara, Calif.). Five micrograms of total RNA from each sample was reverse transcribed using Superscript II (Invitrogen, Carlsbad, Calif.). In vitro transcription to generate biotinylated cRNA was performed using the Bioarray High Yield RNA Transcript Labeling kit (Enzo Diagnostics, Farmingdale, N.Y.). Fifteen micrograms of fragmented cRNA was hybridized for 16 h to mouse MOE430A chips at 45° C. using the Affymetrix 640 hybridization oven, stained, and scanned using the Agilent scanner according to standard Affymetrix protocols. Signal intensities from the Affymetrix “.CEL” files were derived using GeneChip Operating Software v1.2 (Affymetrix, Santa Clara, Calif.), multiplicative model-based expression index (dChip software)¹⁹ and robust multi-array average²⁰. The data discussed in this publication have been deposited in NCBIs Gene Expression Omnibus (GEO) and are accessible through GEO Series accession number GSE4066. Methods employed to determine significant changes in gene expression from the derived signal intensities were Rank Products (RP)₂₁, Significance Analysis of Microarrays (SAM)²², Analysis of Variance (ANOVA), Student's t-test, and fold-change. A list of genes whose expression was altered using each method was created and only genes appearing on all lists were considered significantly altered and reported (Supplementary Table I). Hierarchical clustering of gene expression was performed with dChip software. Samples not exposed to UV were significantly clustered together (p=0.05) versus all other samples suggesting similarities in gene expression among those samples. Significant clustering was also found among samples 6 h after UV and among samples 24 h after UV. Furthermore, significant clustering was found among samples treated with DMSO and among those treated with AG825. Scatter plots were generated using GeneChip Operating Software. Self-Organizing Maps were generated using GeneCluster 2.0 software²³. Grouping of genes into biological processes was performed using NetAffx Analysis Center and literature searches. Genes were mapped into biological networks with Pathways Analysis software (Ingenuity Systems, Mountain View, Calif.). Promoter Analysis and Interaction Network Toolset v3.3 (PAINT) was used to scan sequences up to 2000 base pair upstream of genes with altered expression to search for transcription factor binding sequences or transcriptional response elements (TRE) using a 0.95 core similarity threshold²⁴. The clustering option through PAINT was used to visualize overrepresented Gene-TRE networks using only significantly overrepresented TRE (p=0.05).

Real-Time PCR. Selected changes in gene expression were validated using real-time PCR. Sequences supplied by Affymetrix from the probe set of Il1 b (probe: 1449399_a_at), Mmp9 (probe: 1416298_at) and Thbs1 (probe: 1421811_at) were scanned for unique sequences using a stringent blast search (www.ncbi.nlm.nih.gov/BLAST). Sequences spanning exons were selected preferentially for probes and primers for prepared using Assays-by-Design (Applied Biosystems, Foster City, Calif.). Real-time PCR was performed using TaqMan One-Step RT-PCR Master Mix Reagents (Applied Biosystems, Foster City, Calif.) according to the manufacturer's instructions on an ABI Prism 7000 (Applied Biosystems, Foster City, Calif.). The relative efficiency of amplification of the selected gene versus control (GAPDH or actin) was plotted to ensure the slope of total RNA versus ΔCT was less than 0.05. The 2^{-ΔΔC}^{_{T}} was used to determine fold-change differences between samples²⁵.

Proliferation and apoptosis assays. Proliferation, survival and apoptosis were quantified in three replicate experiments. BrdU uptake was measured by a chemiluminescenit BrdU Cell Proliferation ELISA (Roche, Germany). Survival was quantified using 4-methylumbelliferyl heptanoate (MUH, Sigma, St. Louis, Mo.) degradation product fluorescence (ex: 360 nm, em:465 nm) as a marker for cell viability based on the method by Dotsika et al.²⁷ Cells were incubated in 100 μg/ml MUH in PBS for 30 minutes at 37° C. MUH fluorescence from wells lacking cells was subtracted from all samples. Apoptosis was measured using an ssDNA Apoptosis ELISA kit (Chemicon, Temecula, Calif.).

### EXAMPLES

The present invention is described by reference to the following Examples, which are offered by way of illustration and are not intended to limit the invention in any manner. Standard techniques well known in the art or the techniques specifically described below were utilized.

### Example 1

### Inhibition of Erbb2 Suppresses UV-Induced Skin Tumorigenesis

To test our hypothesis that the inhibition of Erbb2 prevents UV-induced skin carcinogenesis, topical administration of the tyrphostin Erbb2 inhibitor AG825 was applied to block the UV-induced activation of Erbb2. The skin tumorigenesis experiment was performed in v-ras^{Ha} transgenic Tg.AC mice because of their enhanced sensitivity to UV-induced skin tumorigenesis (13; 52). Inhibition of Erbb2 prior to UV irradiation blocked the development of more than half of the tumors, with 34 tumors per vehicle-treated mouse and only 15 tumors per inhibitor-treated mouse by the end of the experiment. While the vehicle-treated mice continued to accrue tumors throughout the duration of the experiment, the AG825-treated group reached a plateau in tumor number by 7 weeks after the first UV exposure. By the end of the experiment, mean tumor volume was also 70% less in the AG825-treated group when compared to the vehicle control. Representative tumors from each treatment were examined histologically and all were benign squamous papillomas with similar differentiation status. These data demonstrate that blocking the UV-induced activation of Erbb2 suppresses skin tumorigenesis.

### Example 2

Abrogation of Erbb2 activity blocks DNA synthesis, resulting in S-phase arrest after UV exposure: Effects not manifested until approximately 18 hours after UV exposure.

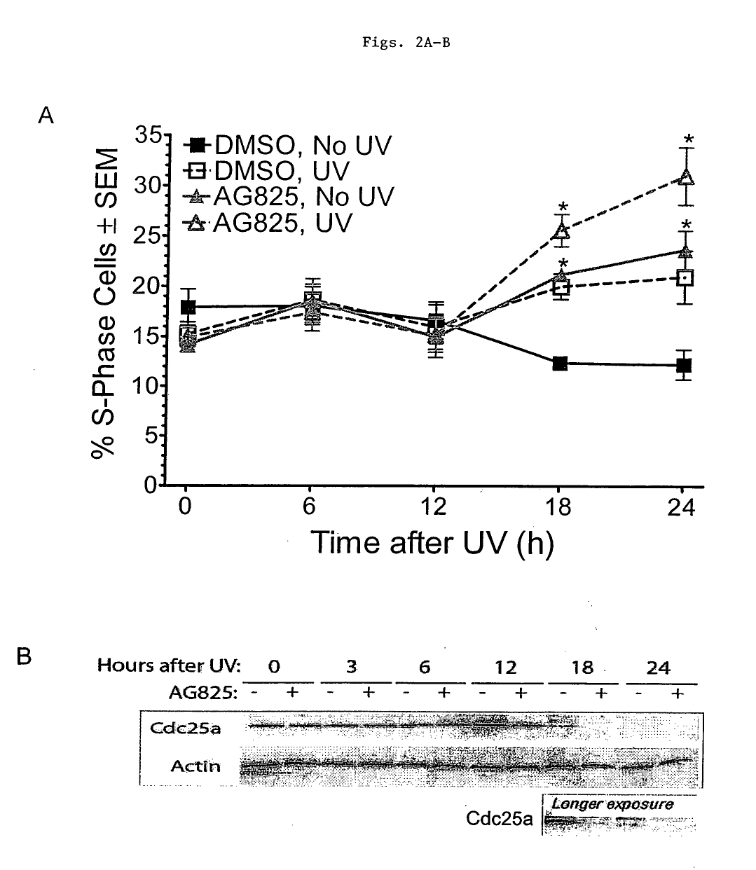

To further investigate Erbb2's effects on cell cycle progression after UV irradiation, DNA synthesis was examined after UV irradiation of cultured primary keratinocytes lacking Erbb2 activity. Both the Erbb2 inhibitor AG825 and transfection of Erbb2-specific siRNA were used to block Erbb2 signaling (Example 9). Bromodeoxyuridine (BrdU) incorporation was significantly reduced in sham-irradiated keratinocytes lacking Erbb2 activity. An even more striking effect was detected after UV irradiation. By 12 h after UV irradiation, BrdU incorporation was reduced to nearly zero in keratinocytes lacking Erbb2 activity while substantial BrDU incorporation occurred in the irradiated controls with intact Erbb2 signaling. Thus, Erbb2 activation was necessary for DNA synthesis following UV irradiation. UV irradiation caused S-phase accumulation of keratinocytes, consistent with previous reports (40). The influence of Erbb2 activation on S-phase progression was determined using both pharmacologic and genetic models to interfere with Erbb2 signaling. Pharmacologic inhibition of Erbb2 prior to UV exposure increased the percentage of cells in S-phase by 10%. This S-phase arrest upon inhibition of Erbb2 began 18 h after UV exposure. siRNA targeting of Erbb2 increased S-phase arrest to a similar extent after UV exposure. Erbb2 was also ablated by Cre recombinase expression in Erbb2^{fl/fl} cultured primary keratinocytes. Genetic ablation of Erbb2 increased UV-induced S-phase arrest by 18% after UV irradiation. These data demonstrate that abrogation of Erbb2 signaling leads to S-phase arrest after-UV irradiation. The discovery that the effect of Erbb2 inhibitors do not manifest until more than 12 hours after treatment, in conjunction with determining S-phase arrest begins 18 hours after UV exposure, suggests that treatment with inhibitors after UV exposure would be effective. Specifically, treatment within the approximately 18 hour window after UV exposure would (FIGS. 2A and B).

It has been found that Erbb2 suppresses UV-induced apoptosis in vitro, although the effect was not large (32). Therefore, the effect of inhibition of Erbb2 on apoptotic cell death was examined in vivo by TUNEL. The percentage of TUNEL-positive basal cells peaked at 18% in vehicle-treated skin at 24 h after UV. Inhibition of Erbb2 did not measurably alter apoptotic cell death in this experiment. These data demonstrate that Erbb2 does not significantly suppress apoptosis in the skin following UV exposure.

### Example 3

### Inhibition of Erbb2 Suppresses UV-Induced Epidermal Hyperplasia and Cell Proliferation

The influence of Erbb2 on epidermal hyperplasia was measured during the first two weeks of the tumor experiment regimen. Little hyperplasia was induced in the first week after UV irradiation, and the effect of the Erbb2 inhibitor on this response was minimal. Following the second UV irradiation, epidermal hyperplasia was significantly suppressed by inhibition of Erbb2. Thus, the UV-induced activation of Erbb2 augments epidermal hyperplasia, a response that becomes more pronounced with multiple UV exposures.

It has been discovered that Erbb2 modulates the expression of genes important in both proliferation and apoptosis following UV exposure in vivo (Example 9). Accordingly, the influence of Erbb2 on both apoptosis and cell proliferation was assessed in order to determine the mechanisms of Erbb2's effect on hyperplasia in the skin after UV irradiation. Erbb2 suppresses UV-induced apoptosis in vitro, although the effect was slight (32). The percentage of TUNEL-positive basal cells was increased at 24 h after UV in vehicle-treated skin, consistent with previous observations in this animal model (13). Inhibition of Erbb2 did not increase apoptotic cell death at this time point. Erbb2 reportedly suppresses keratinocyte differentiation (11), a process leading to keratinocyte death that is mechanistically and morphologically distinct from apoptosis. Inhibition of Erbb2 prior to UV irradiation did not alter the localization or expression of early (keratin 5) or late (loricrin) markers of differentiation. These data demonstrate that Erbb2's influence on hyperplasia and tumor development does not result from the suppression of cell death via apoptosis or terminal differentiation following UV exposure but rather must be a consequence of an effect on cell proliferation. Mean proliferating cell nuclear antigen (PCNA) expression, a marker of cell proliferation, was less in Erbb2 inhibitor treated sham-irradiated skin and in UV-exposed skin when compared to the vehicle-treated controls. Cell cycle analysis revealed that S-phase cells increased upon inhibition of Erbb2 prior to UV irradiation. These data, when combined with our findings of reduced hyperplasia and decreased proliferation, suggested that inhibition of Erbb2 may lead to S-phase arrest after UV irradiation.

### Example 4

### Inhibition of Erbb2 Suppresses UV-Induced Inflammation

Of biological processes important in the response to UV, the immune response category had the second most number of genes whose expression was modulated by Erbb2. Of particular interest was the large number of genes whose expression was lower in Erbb2 inhibitor treated skin 6 h after UV exposure (42 genes) compared to only 3 genes with higher expression (Table I). These results deviated sharply from the general trend of increased gene expression upon inhibition of Erbb2. Cytokines, chemokines, and inflammatory cell markers, many of which are regulated by NF□B and Comp1, were included among these genes. IIIb and Ptgs2 are examples inflammatory mediators whose expression was blocked by inhibition of Erbb2. The UV-induced increase in Il1 b mRNA previously documented and associated with NF□B activity (reviewed in 88), was largely dependent on Erbb2 in both microarray and real-time PCR experiments. Ptgs2, is downstream from NF?B) and increased in keratinocytes after UV (reviewed in 88). Inhibition of Erbb2 suppressed or delayed the UV-induced expression of other inflammatory cell markers such as the mast cell markers Cd48 and Cd53, Sell, the chemoattractant Ptprc (protein tyrosine phosphatase, receptor type, C) and chemokines such as Ccl4, Ccl11, and Ccl12 (also known as MIP-1β, eotaxin, and MCP-5, respectively) 6 h after UV-exposure. By 24 h after UV exposure, the expression of proinflammatory chemokines such as Cxcl2 and Cd44, involved in inflammatory skin disorders, was lower in AG825-treated skin compared to vehicle-treated skin. This analysis predicts an important role for Erbb2 in the potentiation of the immune response following UV exposure.

To further investigate the role of Erbb2 in inflammation following UV exposure, skin-fold thickness as a measure of edema was quantified in mice treated with DMSO or AG825 and exposed to UV. UV exposure maximally increased skin-fold thickness 4 to 5 d post-UV. AG825 suppressed UV-induced edema by 33%, 37% and 64% 4, 5, and 6 d, respectively, after UV exposure. Collectively, these results indicate that activation of Erbb2 by UV augments UV-induced inflammation through NF□B- and Comp1-regulated gene expression.

### Example 5

### Determination of the Effect of Blocking Erbb2 Using CP-724714 on UV-Induced Tumorigenesis

Since abrogation of UV-induced Erbb2 activation decreases tumorigenesis in Tg.AC mice (33), a clinically relevant small molecule inhibitor of Erbb2, CP-724714 (OSI Pharmaceuticals) (38), can be used in SENCAR (SENsitivity to CARcinogenesis) or SSIN inbred SENCAR mice (50), or a different mouse model of skin tumorigenesis. The following procedures can be followed:

Determination of the time of maximum efficacy for the compound. Mice (3 per group at each timepoint) can be given CP-724714 (100 mg/kg, PO) or saline control. Mice can be exposed to 5, 10, or 15 kJ/m² UV or sham irradiated at 0, 0.5, 1, 2, 4, 12, 24, and 48 hours administration of agent. 30 minutes after irradiation, mice can be sacrificed and skin removed. Levels of Erbb2 and p-erbB2 can be measured via Western blot.

Determination of CP-724714's effectiveness of blocking Erbb2 activation after UV exposure. SENCAR mice (3 per group) can be given CP-724714 (100 mg/kg, PO) or saline control. At the time determined in step 1, mice can be exposed to 5, 10, or 15 kJ/m² UV or sham irradiated. Mice can be sacrificed at 0, 0.5, 1, 2, 4, 12, 24, and 48 hours after irradiation and skin removed. Levels of Erbb2 and p-erbB2 can be measured via Western blot.

Determination of the effect of blocking Erbb2 on UV-induced tumorigenesis. 20 SENCAR mice can be given CP-724714 (PO 100 mg/kg) and 20 SENCAR mice can be given saline control. Half of the mice from each treatment group can be exposed to 5, 10 or 15 kJ/m² UV and the other half sham irradiated at the time determined in Step 1. The treatments and exposures can be repeated at one and two weeks later. Tumor number and volume can be counted each week until 20 weeks or other endpoint.

An appropriate dose treatment is 100 mg/kg. Total mice treatments for CP-724,714 are: 96 (Step 1), 96 (Step 2), 120 (Step 3) to give a total of 312 treatments. Thus the amount of compound required is 132×3 mg (+10%), or 1 gram of investigational drug CP-724714 (Pfize)r (stored as powder at 4° C.). 120 mice can be treated with either CP-724714 or DMSO control. CP-724714 can be dissolved in DMSO and can be given at to the mice at 100 mg/kg and dosed in Gulucire 44/14 (Gattefossé, Saint-Priest Cedex, France) at 15 mg/ml. If a mouse weighs approximately 30 grams, it can receive 3 mg CP724714 in 0.2 ml Gelucire. To administer CP-724714 PO, mice can be orally gavaged.

### Example 6

### Erbb2 Promotes Cell Cycle Progression by Maintaining Cdc25a Following UV Irradiation

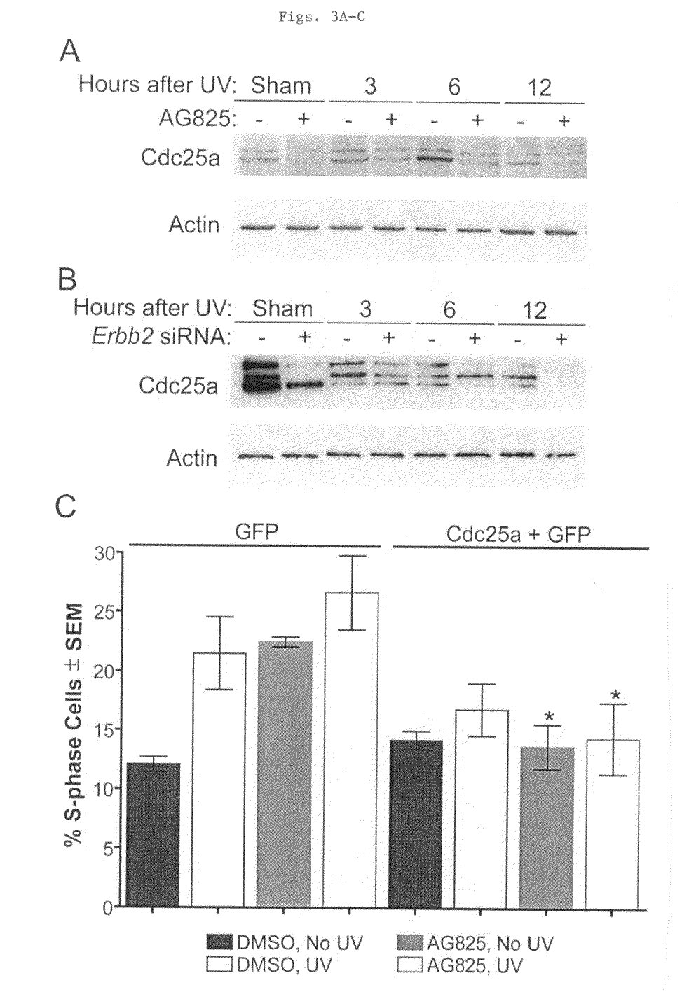

In order to understand how Erbb2 regulates S-phase progression following UV irradiation, signaling pathways known to regulate progression into and through S-phase were examined. As a key mediator of cell cycle progression after UV irradiation, the ATR DNA damage response pathway was examined. Cdc25a is degraded in response to ATR activation and its degradation, in turn, triggers S-phase arrest (15; 30; 34). AG825 or Erbb2-specific siRNA pretreatment decreased Cdc25a immunoreactivity after UV when compared to the appropriate UV-exposed controls (FIG. 3A-B). The decrease in Cdc25a was observed in both sham- and UV-irradiated cells at all time points (FIG. 3A-B). Decreased levels of Cdc25a were associated with increased laddering on the immunoblots, consistent with ubiquitin conjugation of Cdc25a (FIG. 3A-B). The decrease in Cdc25a upon inhibitor treatment occurred prior to the cessation of DNA synthesis at 12 h and S-phase arrest at 18 h. The effect of abrogation of Erbb2 on Cdc25a occurred post-transcriptionally, since no decrease in Cdc25a mRNA was detected in our microarray analysis using the Erbb2 inhibitor (32). Loss of Cdc25a was accompanied by increased Cdc25a phosphorylation, consistent with increased Chk1/2 activity in the absence of Erbb2 signaling. In contrast to the effect of Erbb2 on Cdc25a, Cdc25b and Cdc25c were not significantly decreased upon abrogation of Erbb2 and UV irradiation.

To confirm that the degradation of Cdc25a was the cause of S-phase arrest in cells lacking Erbb2 activity, keratinocytes were cotransfected with a Cdc25a expression vector (18) and a green fluorescent protein (GFP) vector, treated with the Erbb2 inhibitor or vehicle alone, and the cell cycle assessed after UV exposure. Inhibition of Erbb2 prior to UV exposure resulted in the accumulation of keratinocytes in S-phase (FIG. 3C, GFP-only bars on left). Ectopic Cdc25a expression completely blocked this S-phase arrest (FIG. 3C, right-hand bars). Thus, Cdc25a degradation upon abrogation of Erbb2 signaling increases S-phase arrest. These data (FIG. 3A-C) are consistent with both Erbb2-dependent and Erbb2-independent mechanisms regulating Cdc25a degradation and S-phase arrest after UV irradiation.

### Example 7

### Erbb2 Suppresses Chk1 Activation Through a PI3K/Akt-Dependent Mechanism

Phosphorylation of Cdc25a by ATR-activated Chk1 triggers the degradation of Cdc25a. Surprisingly given our results, abrogation of Erbb2 signaling did not affect ATR activity in sham-irradiated or UV-exposed cells (FIG. 4A). However, Chk1 activation, as shown by phosphorylation on Ser²⁹⁶, was slightly increased in both sham- and UV-irradiated keratinocytes lacking Erbb2 activity (FIG. 4B, 0 and 3 h). These results indicate that Erbb2's effects occur downstream from ATR. In cells lacking the tumor suppressor PTEN, AKT can phosphorylate Chk1 at an inhibitory site and block its activation by ATR (22; 26). Consequently, we hypothesized that Erbb2 activation of PI3K/Akt signaling inhibits Chk1 activation. We discovered that the UV-induced activation of PI3K/AKT in primary keratinocytes is largely dependent on Erbb2, as is constitutive Akt activation in unirradiated cells. Both AG825 and Erbb2-specific siRNA reduced Akt activity, as measured by its phosphorylation on immunoblot, in sham-irradiated keratinocytes and at 30 minutes post-UV (FIG. 4C). In addition, inhibition of either PI3K or Akt increased S-phase arrest after UV irradiation, also consistent with our hypothesis (FIG. 4D). From these results, we hypothesized that UV-induced Erbb2 activation dampens the activation of the S-phase ATR checkpoint through a PI3K/Akt-dependent inhibitory phosphorylation of Chk1. Phosphorylation of Chk1 on Ser²⁸⁰ was reduced by more than 50% in sham- and UV-irradiated cells upon ablation of Erbb2 signaling (FIG. 4E). These results demonstrate a novel mechanism by which Erbb2 suppresses activation of the ATR-Chk1 S-phase checkpoint. Interestingly, ablation of Erbb2 signaling also increased the activation of Chk2 upon UV irradiation (FIG. 4B). The mechanism for Erbb2's effect on Chk2 is unclear.

### Example 8

### Inhibition of Erbb2 Signaling Augments Cell Cycle Arrest Induced by the ATR Activator 5-Fluorouracil

Common chemotherapeutic agents induce DNA damage and mutations (37). The ATR DNA damage response pathway is activated by some of these agents as well. We hypothesized that Erbb2 might increase resistance to chemotherapeutic agent induced S-phase arrest by blocking the degradation of Cdc25a after DNA damage. Chemotherapeutic agents such as 5-fluorouracil (5-FU) and camptothecin (3, 15) activate the ATR DNA damage cell cycle checkpoint (2, 9) and cause S-phase arrest in keratinocytes. Consistent with our hypothesis, inhibition of Erbb2 prior to or concurrent with 5-FU treatment further increased S-phase arrest (FIG. 4F). These results suggest treatment with Erbb2 inhibitor as a mechanism to overcome resistance of HER2-overexpressing cancer to chemotherapy.

### Example 9

### Erbb2 Regulates the Expression of Genes Important in Many Biological Processes

In order to determine the biological significance of Erbb2 activation in UV-exposed skin, GO mining and other search strategies were performed to associate changes in gene expression with biological processes. Statistical analysis was performed on the probability of the number of genes occurring in these processes. Changes in the expression of 158 genes with a function in metabolism accounted for the largest percentage (44%) of the changes. Many genes important in the immune response (65 genes), cell communication (65 genes), cancer (55 genes), adhesion and migration (47 genes), development (55 genes), proliferation (36 genes), and apoptosis (25 genes) were also regulated by AG825 (Table I). Six genes relating to pigmentation, mainly melanin biosynthesis, were revealed by the microarray analysis, comprising one-third (6 of 18) of the genes in the Mouse Genome Informatics database known to be involved in pigmentation.

TABLE I
Inhibition of Erbb2 and UV exposure alters the expression
of genes involved in various biological processes.

| | Sham | | 6 h post-UV | | 24 h post-UV | | | |
|---|---|---|---|---|---|---|---|---|
| Biological Process | ↓ | ↑ | ↓ | ↑ | ↓ | ↑ | Total^{a} | |
| Proliferation | 3^{b} | 2 | 6 | 21 | 3 | 6 | 36 | (10%)^{c} |
| Apoptosis | 2 | 2 | 6 | 5 | 4 | 6 | 25 | (7%) |
| Immune Response | 0 | 8 | 42 | 3 | 14 | 4 | 65 | (18%) |
| Adhesion and | 0 | 7 | 13 | 14 | 14 | 4 | 47 | (13%) |
| Migration | | | | | | | | |
| Cancer | 3 | 5 | 12 | 25 | 7 | 8 | 55 | (15%) |
| Metabolism | 10 | 9 | 23 | 63 | 24 | 43 | 158 | (44%) |
| Development | 2 | 6 | 8 | 26 | 12 | 4 | 55 | (15%) |
| Pigment | 3 | 0 | 0 | 5 | 0 | 1 | 6 | (2%) |
| Biosynthesis | | | | | | | | |
| Cell | 1 | 6 | 18 | 18 | 20 | 5 | 65 | (18%) |
| Communication | | | | | | | | |

| | | | | | | | |
|---|---|---|---|---|---|---|---|
| Total Genes | 14 | 25 | 85 | 133 | 59 | 68 | 361 |

^{a}Note that since the expression of some genes is changed at multiple times, the total may not reflect the sum of the individual timepoints.
^{b}Number of genes increased (↑) or decreased (↓) in AG825-treated samples when compared to the vehicle alone.
^{c}Numbers in parentheses indicate the percentage of the total number of genes altered by Erbb2 for each biological process.

Changes in gene expression over time were clustered and trends in the data graphed in self-organizing maps (SOMs), revealing distinct patterns of gene expression upon inhibition of Erbb2. Several prominent patterns were evident including delayed up- or down-regulation or a more sustained decrease or increase in gene expression after UV in the inhibitor-treated skin compared to the corresponding control. The expression of many cell cycle regulatory genes such as Ccna2, Ccnb1 and Ccnb2 (Cyclin A2, B1 and B2), demonstrated a lesser responsiveness to UV after AG825 treatment. Genes associated with inflammation such as Il1 b (Interleukin-1β), Ptgs2/COX2 (Prostaglandin-endoperoxidase synthase 2/Cyclooxygenase-2), Sell (lymphocyte adhesion modulator L-selectin), Ccl4, Cxcl2, and Cd44, demonstrated an Erbb2-dependent induction after UV. The expression of genes with altered expression in tumorigenesis such as Hifla (hypoxia inducible factor I, a subunit), Sppl (secreted phosphoprotein 1), and Ghr (growth hormone receptor) followed this pattern as well. The microarray data revealed that 54 genes implicated in cancer were modulated by Erbb2, most with reduced expression after Erbb2 inhibition. The vast majority of genes were not significantly altered by inhibitor treatment or UV exposure.

While genes involved in many biological processes generally had diverse expression profiles, two consistent trends appeared. The most predominant of these was the large number of inflammation-associated genes whose UV-induced expression was blocked by the Erbb2 inhibitor. The second most prevalent pattern was a lesser decrease of proliferation- and apoptosis-associated genes after UV in samples treated with the Erbb2 inhibitor. These data suggest Erbb2 modulates gene expression in the skin after UV which favors inflammation and plays a role in proliferation and apoptosis.

### Example 10

### Erbb2-Regulated Genes have Binding Sites for Several Transcription Factors

PAINT analysis of the microarray data was used to identify the transcription factors responsible for Erbb2's effects on gene expression. PAINT analyzed the TRE occurrences for over- or under-representation in our gene lists compared to the frequency predicted by examining all genes present on the microarray. Four TREs were identified as being significantly over-represented on a subset of 29 genes identified by microarray analysis. Clustering of the data linked Erbb2-regulated genes with these particular transcription factors and indicated their relatedness. Tall (T-cell acute lymphocytic leukemia 1) (p<0.05), which was found upstream of the cancer-associated genes Plala (phospholipase A1 member A) and Tdel (tumor differentially expressed 1), was the most closely related transcription factor to NF?B. Consistent with this result, immunoblotting revealed that Tall protein was decreased by 70% upon inhibition of Erbb2 in sham-irradiated mouse skin. A binding site for the transcription factor NF?B (p=0.01), known for its role in inflammation, proliferation and apoptosis (reviewed in 8, 109), was significantly over-represented upstream of seven genes whose expression was regulated by Erbb2. Several of these genes have a role in inflammation, including Il1 b, Tdel, Ptgs2, H2-T23 (histocompatibility 2, T region locus 23), Cxcl2 and Cxc110. Consistent with the PAINT analysis, immunoblotting of sham-irradiated mouse skin revealed a 65% decrease in NF?B protein upon inhibition of Erbb2. A Comp1 binding site was significantly over-represented (p=0.01) upstream of 16 genes, 9 which were related to inflammation. These included Hrnr (Hornerin), ligpl (Interferon inducible GTPase 1), Tral (Tumor rejection antigen gp96), Tgtp (T-cell specific GTPase), Cc14, Pfc (Properdin factor, complement), Mgl1 (macrophage galactose N-acetyl-galactosamine specific lectin 1), Lcp2 (lymphocyte cytosolic protein 2) and H2-Eb1 (histocompatibility 2, class II antigen Eβ). These data suggest a role for Comp1, a little-investigated transcription factor, in the Erbb2-regulated, UV-induced inflammatory response. FoxJ2 (Forkhead box J2, p=0.02) was the least closely related and found upstream of Erbb2-regulated genes involved in diverse processes. In summary, our analysis indicated that the transcription factors NF?B, Tall, and Comp1 may lead to increased expression of proinflammatory genes upon UV exposure.

### Example 11

### Erbb2 Regulates the Transcription of Many Genes Following UV Irradiation

The influence of Erbb2 on gene expression following UV exposure was assessed using microarray analysis. Skin from mice treated with the Erbb2 inhibitor AG825 or the vehicle alone prior to each of two UV or sham exposures was harvested either 6 or 24 h post-UV. Mice were exposed to UV twice because previous results indicated the influence of Erbb receptors on the response of the skin to UV increases with multiple exposures (22). A measure of the inter-animal variation among the mice in each group is revealed by scatter plots comparing gene expression in two mice from the same group. In two randomly selected vehicle-treated and sham-irradiated mice, 8 genes out of approximately 13,000 detected (<0.1%) were expressed with more than a 5-fold difference between them. Similar results were observed with UV-exposed or AG825-treated skin (data not shown). In contrast to the comparison of gene expression between samples of the same group, a plot of AG825-treated skin compared to vehicle-treated skin produced a much wider scatter. Many of the data points are shifted upwards indicating that inhibition of Erbb2 more often increased rather than decreased gene expression. This trend occurred in both sham- and UV-irradiated skin. Regardless of inhibitor treatment, the scatter plot of UV-exposed compared to sham-irradiated skin was much broader than any other plot.

Further analysis of the microarray data produced a list of 361 genes whose expression changed significantly upon abrogation of Erbb2 activity. The expression of 61% of these genes was increased upon Erbb2 inhibition. Inhibition of Erbb2 without UV irradiation altered the expression of 39 genes, with 64% of these increased upon AG825 treatment. Inhibition of Erbb2 prior to UV exposure altered the expression of 218 (61% with increased expression) and 127 genes (54% with increased expression) at 6 and 24 h, respectively. Thus, Erbb2 activation more frequently decreased rather than increased gene expression after UV.

### Example 12

### Real-Time PCR Validates the Microarray Analysis

The microarray results were validated using real-time PCR of selected genes whose expression was altered at least two-fold after AG825 treatment, consistent with genes such as Il1 b expression, similar in sham-irradiated AG825- and vehicle-treated skin, was increased to a greater extent after UV exposure in the vehicle-treated compared to AG825-treated samples in both the real-time PCR (6-fold greater increase) and the microarray experiments (16-fold greater increase). Analysis of the Affymetrix data for Mmp9 (Matrix metalloproteinase 9) detected 3.8- and 2-fold increases in Mmp9 expression 24 h after UV exposure of vehicle- and inhibitor-treated skin, respectively. Consistent with these results, qPCR found 3.2- and 1.3-fold increases in Mmp9 expression in the vehicle- and AG825-treated mice 24 h after UV exposure, respectively. Both the Affymetrix probe and real-time PCR for Thbs1 (Thrombospondin 1) detected a significant 2-fold higher Thbs1 expression in AG825-treated and sham-irradiated skin when compared to the corresponding control but similar Thbs1 expression in vehicle- and inhibitor-treated mice at 24 h post-UV. In addition, real-time PCR of Padi3 (peptidyl arginine deiminase type III), Chi313 (chitinase 3-like 3) and Socs3 (Suppressor of cytokine signaling 3) yielded statistically similar results compared to the microarray data. These data validate the microarray analysis for identification of altered gene expression.

### Example 13

### Erbb2 Suppresses Apoptosis after UV Exposure

While EGFR has been shown to decrease apoptosis in the skin after UV (22), it was expected that Erbb2 may also play a role in modulating apoptosis in the skin after UV. Microarray analysis demonstrated that Erbb2 modulates the expression of 25 apoptosis-associated genes, most of them after UV exposure (Table I). Genes that were decreased to a lesser extent after AG825 included pro-apoptotic genes such Pawr (PRKC, apoptosis, WT1 regulator) and Sox4 (SRY-box containing gene 4), as well as markers of apoptosis such as Pdcd4 (programmed cell death 4). The anti-apoptotic genes Ghr and Ptgs2 were suppressed by the Erbb2 inhibitor. These changes in gene expression predicted increased apoptosis after UV in the absence of Erbb2 signaling.

To investigate the role of Erbb2 in apoptosis following UV exposure, keratinocyte survival was measured in AG825-treated or Erbb2 siRNA-transfected cells and controls. As expected, apoptosis increased in all groups after UV exposure, as detected by ssDNA quantification. AG825- and DMSO-treated keratinocytes exhibited similarly increased ssDNA between 6 and 12 h post-UV. However, by 24 h after UV-exposure, apoptosis was significantly increased in the AG825-treated compared to DMSO-treated cells. Similar results were obtained in keratinocytes transfected with Erbb2-targeted siRNA. Taken together, these results demonstrate that Erbb2 suppresses UV-induced apoptosis.

### Example 14

### Egfr and Erbb2 have Distinct Mechanisms for Affecting Cell Cycle

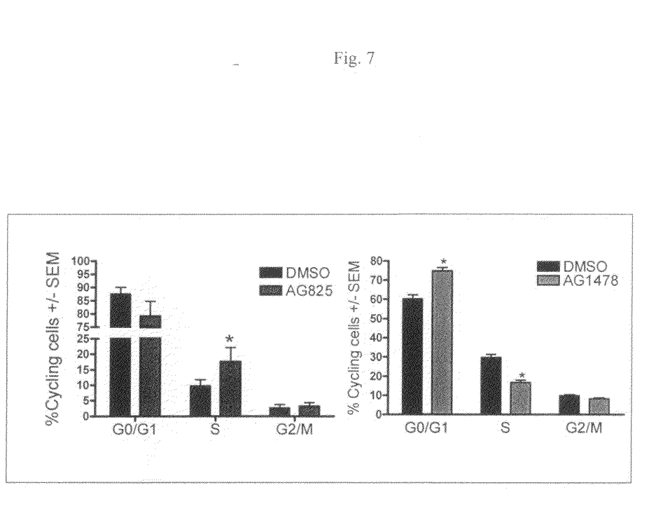

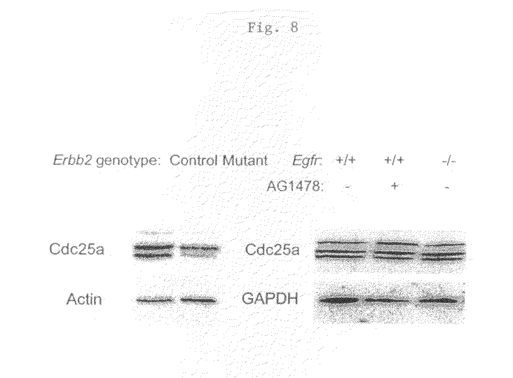

It was previously documented that inhibition of EGFR reduces skin tumorigenesis in a mouse model (22). It has been discovered that inhibition of both EGFR and Erbb2 together results in better suppression of UV-induced skin tumorigenesis than does inhibition of either receptor alone (FIG. 6). In addition, investigation of the role of EGFR in UV-induced skin cancer has revealed that EGFR stimulates cell proliferation and suppresses apoptosis during UV-induced skin cancer. Surprisingly, however, EGFR and Erbb2 have distinct mechanisms for their effects on the cell cycle. As reported herein, inhibition (FIG. 7) or genetic deletion of EGFR results in a G₁ arrest, rather than the S-phase arrest resulting from ablation of Erbb2 (FIG. 7). S-phase arrest in cells lacking Erbb2 is associated with and dependent on decreased Cdc25a (FIG. 8, left panel). However, Cdc25a is not decreased in Egfr null or EGFR inhibitor treated keratinocytes (FIG. 8, right panel). Thus, while it is true that pan-Erbb inhibitors would have the best anti-cancer potential, our research demonstrates that EGFR and Erbb2 inhibitors have very different effects on the response of the skin to UV.

### Example 15

### Other Cell Cycle Related Targets of Erbb2

Preliminary experiments have shown that Erbb2 suppresses p27 after UV irradiation. Experimental results reported herein document Erbb2 regulation of p27 in keratinocytes. Additional immunoblotting data for several cyclin-dependent kinase inhibitors was collected. These experiments eliminated several signaling pathways; including p21, p53, and p18; as responsible for the S-phase arrest occurring upon ablation of Erbb2 signaling. These results suggest that inhibition of Erbb2 reduces mutagenesis. It is also possible to systematically test the influence of Erbb2 on critical cell cycle regulatory molecules. This can be done with the use of a cell cycle array (Superarray, Bioscience Corp.) to examine the influence of Erbb2 on 84 genes that both positively and negatively regulate the cell cycle and cell cycle checkpoints.

### Example 16

### The Effects of Genetic Ablation of Erbb2 on Response to UV

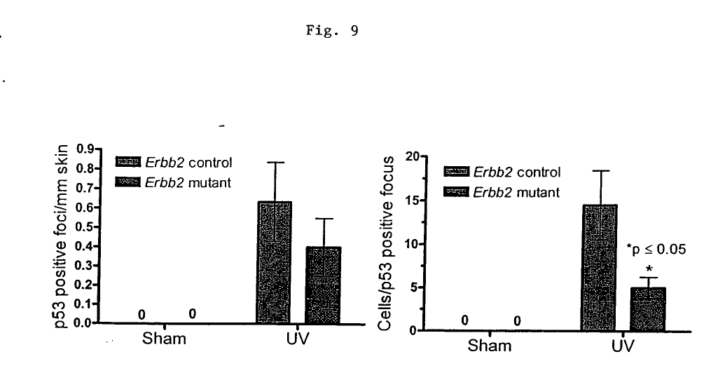

In addition to the experiments that employ an Erbb2 inhibitor, the effects of genetic ablation of Erbb2 on the response of the skin to UV were examined. Trp53 mutations were assessed in UV-exposed skin from skin-targeted Erbb2 mutant and control mice. Trp53 mutations are the most common mutations detected in UV-induced skin tumors. Fewer and smaller p53-positive foci were detected in chronically UV-exposed skin lacking Erbb2 expression when compared to UV-exposed control skin (FIG. 9).

### Example 17

### Effect of Genetic Ablation of Erbb2 on Cell Cycle

In addition to investigating the mechanisms through which Erbb2 suppresses cell cycle arrest following UV irradiation, it was demonstrated that genetic ablation of Erbb2 results in S-phase arrest and reduces Cdc25a immunoreactivity (FIG. 8, left panel). Also consistent with our data showing that Erbb2 impacts the cell cycle checkpoint downstream from ATR, we have demonstrated that Erbb2 does not alter the activation of ATR/M protein kinases.

### Example 18

### Biological Significance of Erbb2 During Tumor Angiogenesis

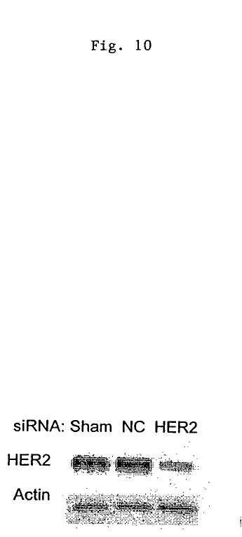

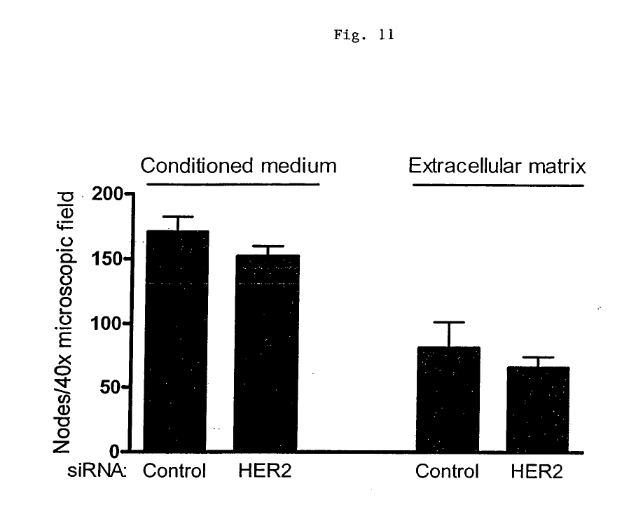

The importance of Erbb2 on angiogenesis during progression to malignancy was examined. HER2 (the human form of Erbb2) was modulated in a squamous cell carcinoma line using HER2-targeted siRNA (FIG. 10). The effects of conditioned medium and extracellular matrix from carcinoma cells with or without HER2 expression were determined in an in vitro angiogenesis assay (FIG. 1). In these experiments, tube formation was reduced when endothelial cells were incubated with conditioned medium from carcinoma cells lacking HER2 expression or when they were cultured on extracellular matrix laid down by carcinoma cells that did not express HER2 (FIG. 11).

### Example 19

### Suppression of UV-Induced Skin Carcinogenesis Upon Inhibition of an Erbb2 Dimerization Partner

Upon activation, Erbb2 heterodimerizes with either EGFR or Erbb3 to transduce signals. Support for the hypothesis that Erbb2 increases skin carcinogenesis comes from our investigations of the functions of its dimerization partner EGFR. A tumor study was conducted to assess the role of EGFR in the clonal expansion of transformed cells to form benign tumors in the skin of v-ras^{Ha} transgenic Tg.AC mice. Inhibition of EGFR with the tyrphostin inhibitor AG1478 decreased the number of tumors that developed by about 50% in UV-exposed v-rasHa transgenic mice. Mean tumor volume was 80% less upon inhibition of EGFR-mediated signaling when compared to, UV-exposed and vehicle-treated controls. These results indicate that Erbb receptors contribute to UV-induced skin carcinogenesis.

### Example 20

### Models for the Abrogation of Erbb2 Activity Through Genetic and Pharmacological Means

Several methods were developed to regulate Erbb2 expression and activity both in vitro and in vivo. First, the Erbb2 tyrphostin inhibitor AG825 blocked the activation of Erbb2 by UV, both in vivo and in vitro. AG825 inhibited the UV-induced phosphorylation of Erbb2 at Tyr¹²⁴⁸ in cultured keratinocytes without altering expression levels of Erbb2. Similar results were obtained in epidermal protein extracts from mice treated with AG825. Since activated Erbb2 dimerizes with and transphosphorylates its partners EGFR and Erbb3, UV-induced phosphorylation of EGFR-Tyr⁹⁹² and Erbb3-Tyr¹²⁸⁹ was blocked by AG825 and by genetic ablation of Erbb2. Surprisingly, however, AG825 did not block EGFR phosphorylation at Tyr¹¹⁷³ after UV, indicating some specificity of Erbb2 signaling upon activation. Consistent with this hypothesis, immunoprecipitation experiments revealed heterodimerization of Erbb2 with both EGFR and Erbb3. The Erbb2 inhibitor AG825 specifically targets the receptor Erbb2, as indicated by the more than 50-fold higher IC₅₀ for EGFR and more than 100-fold higher IC₅₀ for other protein targets such as the platelet-derived growth factor receptor kinase, insulin-like growth factor I receptor kinase, and Abl. Collectively, these experiments demonstrate the ability to specifically inhibit the UV-induced activation of Erbb2 and supports that Erbb2 plays an important role in UV-induced skin cancer.

Two additional in vitro and in vivo models, siRNA targeting of Erbb2 and skin-targeted Erbb2 null mice, were used to ablate Erbb2 signaling. Erbb2-targeted siRNA decreased Erbb2 expression, but not EGFR or Erbb3 expression, in keratinocytes were developed. A skin-targeted deletion of Erbb2 was also created because of the early lethality of Erbb2 null mice. Skin-targeted Erbb2 null mice that lack cutaneous expression of Erbb2 were created by breeding Erbb2^{fl/fl} and keratin 14 (K14) promoter-driven Cre recombinase transgenic mice. This novel mouse model can be used to further study Erbb2 function in Erbb2 and HER2 over expressing skin abnormalities. To obtain skin-targeted Erbb2 null mice, homozygous floxed Erbb2 mice were mated with skin-targeted K14 promoter-driven Cre-recombinase mice (The Jackson Laboratory), backcrossed, to produce Cre recombinase^{+/-} Erbb2^{flfl} mice. Skin from Erbb2 mutant, hemizygous and control mice was removed and immunoblotted for Erbb2 and actin. The efficiency of targeting of Erbb2 was determined by immunoblotting (FIG. X), which reveals a 96% reduction of Erbb2 immunoreactivity in the skin. These mice are healthy and viable into adulthood and after UV irradiation. Littermates that express the Cre recombinase transgene and are hemizygous for the Erbb2 allele (Erbb2^{fl/wt)}(Fig. X), have Erbb2 levels that are approximately half that of normal physiological levels in the skin.

From the foregoing it will be seen that this invention is one well adapted to attain all ends and objectives herein-above set forth, together with the other advantages which are obvious and which are inherent to the invention. Since many possible embodiments may be made of the invention without departing from the scope thereof, it is to be understood that all matters herein set forth are to be interpreted as illustrative, and not in a limiting sense. While specific embodiments have been shown and discussed, various modifications may of course be made, and the invention is not limited to the specific forms or arrangement of parts and steps described herein, except insofar as such limitations are included in the following claims. Further, it will be understood that certain features and subcombinations are of utility and may be employed without reference to other features and subcombinations. This is contemplated by and is within the scope of the claims.

### LIST OF REFERENCES

1. Afaq, F., Adhami, V. M., and Mukhtar, J. (2005). Photochemopreventions of ultraviolet B signaling and photocarcinogenesis. Mutant Res, 571: 153-173.
2. Afonja, O., Juste, D., Das, S., Matsuhashi, S., Samuels, H. H. (2004): Induction of PDCD4 tumor suppressor gene expression by RAR agonists, antiestrogen and HER-2/neu antagonist in breast cancer cells. Evidence for a role in apoptosis, Oncogene, 23:8135-8145
3. Agner, J., Falck, J., Lukas, J., and Bartek, J. (2005). Differential impact of diverse anticancer chemotherapeutics on the Cdc25A-degradation checkpoint pathway. Exp. Cell Res. 302, 162-169.
4. Baadsgaard, O., Salvo, B., Mannie, A., Dass, B., Fox, D. A., Cooper, K. D. (1990): In vivo ultraviolet-exposed human epidermal cells activate T suppressor cell pathways that involve CD4+ CD45RA+suppressor-inducer T cells, J. Immunol., 145:2854-2861
5. Bacus, S. S., Altomare, D. A., Lyass, L., Chin, D. M., Farrell, M. P., Gurova, K., Gudkov, A., and Testa, J. R. (2002). AKT2 is frequently upregulated in HER-2/neu-positive breast cancers and may contribute to tumor aggressiveness by enhancing cell survival. Oncogene 21, 3532-3540.
6. Barnes, P. J., Karin, M. (1997): Nuclear factor-kappaB: a pivotal transcription factor in chronic inflammatory diseases, N. Engl. J. Med., 336:1066-1071
7. Bartkova, J., Horejsi, Z., Koed, K., Kramer, A., Tort, F., Zieger, K., Guldberg, P., Sehested, M., Nesland, J. M., Lukas, C., Orntoft, T., Lukas, J., and Bartek, J. (2005). DNA damage response as a candidate anti-cancer barrier in early human tumorigenesis. Nature 434, 864-870.
8. Bell, S., Degitz, K., Quirling, M., Jilg, N., Page, S., Brand, K. (2003): Involvement of NF-kappaB signalling in skin physiology and disease, Cell Signal, 15:1-7
9. Blomberg, I. and Hoffmann, I. (1999). Ectopic expression of Cdc25A accelerates the G(1)/S transition and leads to premature activation of cyclin E- and cyclin A-dependent kinases. Mol. Cell. Biol. 19, 6183-6194.
10. Bol, D., Kiguchi, K., Beltran, L., Rupp, T., Moats, S., Gimenez-Conti, I., Jorcano, J., and DiGiovanni, J. (1998). Severe follicular hyperplasia and spontaneous papilloma formation in transgenic mice expressing the neu oncogene under the control of the bovine keratin 5 promoter. Mol. Carcinog. 21, 2-12.
11. Bortul, R., Tazzari, P. L., Billi, A. M., Tabellini, G., Mantovani, I., Cappellini, A., Grafone, T., Martinelli, G., Conte, R., and Martelli, A. M. (2005). Deguelin, A PI3K/AKT inhibitor, enhances chemosensitivity of leukaemia cells with an active PI3K!AKT pathway. Br. J. Haematol. 129, 677-686.
12. Box, A. H. and Demetrick, D. J. (2004). Cell cycle kinase inhibitor expression and hypoxia-induced cell cycle arrest in human cancer cell lines. Carcinogenesis 25, 2325-2335.
13. Chang, A. A., Van Waes, C. (2005): Nuclear factor-KappaB as a common target and activator of oncogenes in head and neck squamous cell carcinoma, Adv. Otorhinolaryngol, 62:92-102
14. Chiariello, M., Marinissen, M. J., Gutkind, J. S. (2000): Multiple mitogen-activated protein kinase signaling pathways connect the cot oncoprotein to the c-jun promoter and to cellular transformation, Mol. Cell. Biol., 20:1747-1758
15. Cliby, W. A., Lewis, K. A., Lilly, K. K., and Kaufmann, S. H. (2002). S phase and G2 arrests induced by topoisomerase I poisons are dependent on ATR kinase function. J. Biol. Chem. 277, 1599-1606.
16. Coffer, P. J., Burgering, B. M., Peppelenbosch, M. P., Bos, J. L., and Kruijer, W. (1995). UV activation of receptor tyrosine kinase activity. Oncogene 11, 561-569.
17. Condorelli, G. L., Facchiano, F., Valtieri, M., Proietti, E., Vitelli, L., Lulli, V., Huebner, K., Peschle, C., Croce, C. M. (1996): T-cell-directed TAL-1 expression induces T-cell malignancies in transgenic mice, Cancer Res., 56:5113-5119
18. Costoya, J. A., Finidori, J., Moutoussamy, S., Searis, R., Devesa, J., Arce, V. M. (1999): Activation of growth hormone receptor delivers an antiapoptotic signal: evidence for a role of Akt in this pathway, Endocrinology, 140:5937-5943
19. Dazard, J. E., Gal, H., Amariglio, N., Rechavi, G., Domany, E., Givol, D. (2003): Genome-wide comparison of human keratinocyte and squamous cell carcinoma responses to UVB irradiation: implications for skin and epithelial cancer, Oncogene, 22:2993-3006
20. De Potter, I. Y., Poumay, Y., Squillace, K. A., and Pittelkow, M. R. (2001). Human EGF receptor (HER) family and heregulin members are differentially expressed in epidermal keratinocytes and modulate differentiation. Exp. Cell Res. 271, 315-328.
21. Do, N.Y., Lim, S.C., Im, T. S. (2004): Expression of c-erbB receptors, MMPs and VEGF in squamous cell carcinoma of the head and neck, Oncol. Rep., 12:229-237
22. El Abaseri, T. B., Fuhrman, J., Trempus, C., Shendrik, I., Tennant, R. W., and Hansen, L. A. (2005). Chemoprevention of UV light-induced skin tumorigenesis by inhibition of the epidermal growth factor receptor. Cancer Res. 65, 3958-3965.
23. El Abaseri, T. B., Putta, S., and Hansen, L. A. (2006). Ultraviolet irradiation induces keratinocyte proliferation and epidermal hyperplasia through the activation of the epidermal growth factor receptor. Carcinogenesis 27, 225-231.
24. Enk, C. D., Shahar, I., Amariglio, N., Rechavi, G., Kaminski, N., Hochberg, M. (2004): Gene expression profiling of in vivo UVB-irradiated human epidermis, Photodermatol Photoimmunol Photomed., 20:129-137
25. Falck, J., Mailand, N., Syljuasen, R. G., Bartek, J., and Lukas, J. (2001). The ATM-Chk2-Cdc25A checkpoint pathway guards against radioresistant DNA synthesis. Nature 410, 842-847.
26. Garcia-Cao, I., Duran, A., Collado, M., Carrascosa, M. J., Martin-Caballero, J., Flores, J. M., Diaz-Meco, M. T., Moscat, J., Serrano, M. (2005): Tumour-suppression activity of the proapoptotic regulator Par4, EMBO Rep., 6:577-583
27. Garcia-Cao, I., Lafuente, M. J., Criado, L. M., Diaz-Meco, M. T., Serrano, M., Moscat, J. (2003): Genetic inactivation of Par4 results in hyperactivation of NF-kappaB and impairment of JNK and p38, EMBO Rep., 4:307-312
28. Grimbaldeston, M. A., Finlay-Jones, J. J., and Hart, P. H. (2006). Mast cells in photodamaged skin: what is their role in skin cancer? Photochem Photobiol Sci, 5: 177-183.
29. Groves, R. W., Mizutani, H., Kieffer, J. D., and Kupper, T. S. (1995). Inflammatory skin disease in transgenic mice that express high levels of interleukin 1 alpha in basal epidermis. Proc Natl Acad Sci USA, 92: 11874-11878.
30. Hassepass, I., Voit, R., and Hoffmann, I. (2003). Phosphorylation at serine 75 is required for UV-mediated degradation of human Cdc25A phosphatase at the S-phase checkpoint. J. Biol. Chem. 278, 29824-29829.
31. He, Y. Y., Huang, J. L., and Chignell, C. F. (2004). Delayed and sustained activation of extracellular signal-regulated kinase in human keratinocytes by UVA: implications in carcinogenesis. J. Biol. Chem. 279, 53867-53874.
32. Hennings, H. (1994). Primary culture of keratinocytes from newborn mouse epidermis in medium with lowered levels of Ca²⁺. In: Leigh, I. and Watt, F. M. (EDS.), Keratinocyte Methods. Cambridge University Press, Cambridge, UK, pp. 21-23.
33. Herrlich, P., Sachsenmaier, C., Radler-Pohl, A., Gebel, S., Blattner, C., and Rahmsdorf, H. J. (1994). The mammalian UV response: mechanism of DNA damage induced gene expression. Adv. Enzyme Regul. 34, 381-395.
34. Hirose, Y., Katayama, M., Mirzoeva, 0. K., Berger, M. S., and Pieper, R. O. (2005). Akt activation suppresses Chk2-mediated, methylating agent-induced G2 arrest and protects from temozolomide-induced mitotic catastrophe and cellular senescence. Cancer Res. 65, 4861-4869.
35. Huang, R. P., Wu, J. X., Fan, Y., and Adamson, E. D. (1996). UV activates growth factor receptors via reactive oxygen intermediates. J. Cell Biol. 133, 211-220.
36. Hur, E. H., Hur, W., Choi, J. Y., Kim, I. K., Kim, H. Y., Yoon, S. K., Rhim, H. (2004): Functional identification of the pro-apoptotic effector domain in human Sox4, Biochem Biophys Res. Commun., 325:59-67
37. Impola, U., Uitto, V. J., Hietanen, J., Hakkinen, L., Zhang, L., Larjava, H., Isaka, K., Saarialho-Kere, U.(2004): Differential expression of matrilysin-1 (MMP-7), 92 kD gelatinase (MMP-9), and metalloelastase (MMP-12) in oral verrucous and squamous cell cancer, J. Pathol., 202:14-22
38. Jirmanova, L., Bulavin, D. V., and Fornace, A. J., Jr. (2005). Inhibition of the ATR/Chk1 pathway induces a p38-dependent S-phase delay in mouse embryonic stem cells. Cell Cycle 4, 1428-1434.
39. Kallstrom, H., Lindqvist, A., Pospisil, V., Lundgren, A., and Rosenthal, C. K. (2005). Cdc25A localisation and shuttling: characterisation of sequences mediating nuclear export and import. Exp. Cell Res. 303, 89-100.
40. Karin, M., Cao, Y., Greten, F. R., Li, Z. W. (2002): NF-kappaB in cancer: from innocent bystander to major culprit, Nat. Rev. Cancer, 2:301-310
41. Katoh, M., Katoh, M. (2004): Human FOX gene family (Review), Int. J. Oncol., 25:1495-1500
42. King, F. W., Skeen, J., Hay, N., and Shtivelman, E. (2004). Inhibition of Chk1 by activated PKB/Akt. Cell Cycle 3, 634-637.
43. Koukourakis, M. I., Giatromanolaki, A., Sivridis, E., Simopoulos, C., Turley, H., Talks, K, Gatter, K. C., Harris, A. L. (2002): Hypoxia-inducible factor (HIF1A and HIF2A), angiogenesis, and chemoradiotherapy outcome of squamous cell head-and-neck cancer, Int. J. Radiat. Oncol. Biol. Phys., 53:1192-1202
44. Krahn, G., Leiter, U., Kaskel, P., Udart, M., Utikal, J., Bezold, G., and Peter, R. U. (2001). Coexpression patterns of EGFR, HER2, HER3 and HER4 in non-melanoma skin cancer. Eur. J. Cancer 37, 251-259.
45. Lavker, R. M., Veres, D. A. Irwin, C. J., and Kaidbey, K. H. (1995). Quantitative assessment of cumulative damage from repetitive exposures to suberythemogenic doses of UVA in human skin. Photochem Photobio, 62: 348-352.
46. Lazrak M, Deleuze V, Noel D, Haouzi D, Chalhoub E, Dohet C, Robbins I, Mathieu D: The bHLH TAL-1/SCL regulates endothelial cell migration and morphogenesis, J Cell Sci 2004, 117:1161-1171
47. Lee, C. H. and Chung, J. H. (2001). The hCds1 (Chk2)-FHA domain is essential for a chain of phosphorylation events on hCds1 that is induced by ionizing radiation. J. Biol. Chem. 276, 30537-30541.
48. Lee, C. H., Yu, C. L., Liao, W. T., Kao, Y. H., Chai, C. Y., Chen, G. S., and Yu, H. S. (2004). Effects and interactions of low doses of arsenic and UVB on keratinocyte apoptosis. Chem. Res. Toxicol 17, 1199-1205.
49. Lee, K. M., Lee, J. G., Seo, E. Y., Lee, W. H., Nam, Y. H., Yang, J. M., Kee, S. H., Seo, Y. J., Park, J. K., Kim, C. D., Lee, J. H. (2005): Analysis of genes responding to ultraviolet B irradiation of HaCaT keratinocytes using a cDNA microarray, Br. J. Dermatol., 152:52-59.
50. Lee, M. L., Kuo, F. C., Whitmore, G. A., Sklar, J. (2000): Importance of replication in microarray gene expression studies: statistical methods and evidence from repetitive cDNA hybridizations, Proc. Natl. Acad. Sci. U.S.A., 97:9834-9839.
51. Leroy-Viard, K., Vinit, M. A., Lecointe, N., Jouault, H., Hibner, U., Romeo, P. H., Mathieu-Mahul, D. (1995): Loss of TAL-1 protein activity induces premature apoptosis of Jurkat leukemic T cells upon medium depletion, Embo. J., 14:2341-2349.
52. Ley, K. D. and Ellem, K. A. (1992). UVC modulation of epidermal growth factor receptor number in HeLa S3 cells. Carcinogenesis 13, 183-187.
53. Lin, L., Spoor, M. S., Gerth, A. J., Brody, S. L., Peng, S. L. (2004): Modulation of Th1 activation and inflammation by the NF-kappaB repressor Foxj1, Science, 303:1017-1020.
54. Low, Q. E., Drugea, I. A., Duffner, L. A, Quinn, D. G., Cook, D. N., Rollins, B. J., Kovacs, E. J., DiPietro, L. A. (2001): Wound healing in MIP-1 alpha(−/−) and MCP-1(−/−) mice, Am. J. Pathol., 159:457-463.
55. Mackay, A., Jones, C., Dexter, T., Silva, R. L., Bulmer, K., Jones, A., Simpson, P., Harris, R. A., Jat, P. S., Neville, A. M., Reis, L. F., Lakhani, S. R., O'Hare, M. J. (2003): cDNA microarray analysis of genes associated with ERBB2 (HER2/neu) overexpression in human mammary luminal epithelial cells, Oncogene, 22:2680-2688.
56. Madson, J. G., Lynch, D. T., Tinkum, K. L., Putta, S. K., and Hansen, L. A. (2006). Erbb2 regulates inflammation and proliferation in the skin after ultraviolet irradiation. Am. J. Pathol. 169, 1402-1414.
57. Madson, J. G., Svoboda, J. L., Evans, B. R., Nicolai, J. R., Repertinger, S. K. Trempus, C. S., Tennant, R. W., Hansen, L. A. (2006). Inhibition of Erbb2 suppresses UV-induced skin tumorigenesis through a mechanism independent of EGFR.
58. Mailand, N., Falck, J., Lukas, C., Syljuasen, R. G., Welcker, M., Bartek, J., and Lukas, J. (2000). Rapid destruction of human Cdc25A in response to DNA damage. Science 288, 1425-1429.
59. Maubec, E., Duvillard, P., Velasco, V., Crickx, B., and Avril, M. F. (2005). Immunohistochemical analysis of EGFR and HER-2 in patients with metastatic squamous cell carcinoma of the skin. Anticancer Res. 25, 1205-1210.
60. Maurer, M., Theoharides, T., Granstein, R. D., Bischoff, S.C., Bienenstock, J., Henz, B., Kovanen, P., Piliponsky, A. M., Kambe, N., Vliagoftis, H., Levi-Schaffer, F., Metz, M., Miyachi, Y., Befus, D., Forsythe, P., Kitamura, Y., Galli, S. (2003): What is the physiological function of mast cells? Exp. Dermatol., 12:886-910
61. Menard, S., Pupa, S. M., Campiglio, M., and Tagliabue, E. (2003). Biologic and therapeutic role of HER2 in cancer. Oncogene 22, 6570-6578.
62. Mukhtar, H. and Elmets, C. A. (1996). Photocarcinogenesis: mechanisms, models and human health implications. Photochem. Photobiol. 63, 356-357.
63. Munster P N, Mita M, Britten C, Minton S, Moulder S, Noe D, Roedig B, Denis L, Slamon D, Tolcher A (2004). Phase I and pharmacokinetic (PK) Study of CP-724,714, an oral human epidermal growth factor receptor-2 (HER-2) selective tyrosine kinase inhibitor, J Clin Oncol (Meeting Abstracts), 22:3082.
64. Murakami, T., Fujimoto, M., Ohtsuki, M., Nakagawa, H. (2001): Expression profiling of cancer-related genes in human keratinocytes following non-lethal ultraviolet B irradiation, J. Dermatol. Sci., 27:121-129.
65. Nagata, Y., Lan, K. H., Zhou, X., Tan, M., Esteva, F. J., Sahin, A. A., Klos, K. S., Li, P., Monia, B. P., Nguyen, N. T., Hortobagyi, G. N., Hung, M. C., and Yu, D. (2004). PTEN activation contributes to tumor inhibition by trastuzumab, and loss of PTEN predicts trastuzumab resistance in patients. Cancer Cell 6, 117-127.
66. Neades, R., Cox, L., and Pelling, J. C. (1998). S-phase arrest in mouse keratinocytes exposed to multiple doses of ultraviolet B/A radiation. Mol. Carcinog. 23, 159-167.
67. Nickoloff, B. J., Ben-Neriah, Y., Pikarsky, E. (2005): Inflammation and cancer: is the link as simple as we think? J. Invest. Dermatol., 124:x-xiv.
68. O-charoenrat, P., Rhys-Evans, P. H., Modjtahedi, H., Eccles, S. A. (2002): The role of c-erbB receptors and ligands in head and neck squamous cell carcinoma, Oral. Oncol., 38:627-640.
69. Park, J. S., Jun, H. J., Cho, M. J., Cho, K. H., Lee, J. S., Zo, J. I., and Pyo, H. (2006). Radiosensitivity enhancement by combined treatment of celecoxib and gefitinib on human lung cancer cells. Clin. Cancer Res. 12, 4989-4999.
70. Petrocelli, T., Poon, R., Drucker, D. J., Slingerland, J. M., and Rosen, C. F. (1996). UVB radiation induces 21^{Cip1/WAF1} and mediates G1 and S phase checkpoints. Oncogene 12, 1387-1396.
71. Potter, T., Gohde, W., Wedemeyer, N., and Kohnlein, W. (2000). Keratinocytes exposed to ultraviolet radiation reveal three down-regulated genes with potential function in differentiation and cell cycle control. Radiat. Res. 154, 151-158.
72. Puc, J., Keniry, M., Li, H. S., Pandita, T. K., Choudhury, A. D., Memeo, L., Mansukhani, M., Murty, V. V., Gaciong, Z., Meek, S. E., Piwnica-Worms, H., Hibshoosh, H., and Parsons, R. (2005). Lack of PTEN sequesters CHK1 and initiates genetic instability. Cancer Cell 7, 193-204.
73. Romagnani, P., De Paulis, A., Beltrame, C., Annunziato, F., Dente, V., Maggi, E., Romagnani, S., Marone, G. (1999): Tryptase-chymase double-positive human mast cells express the eotaxin receptor CCR3 and are attracted by CCR3-binding chemokines, Am. J. Pathol., 155:1195-1204.
74. Rosette, C. and Karin, M. (1996). Ultraviolet light and osmotic stress: activation of the JNK cascade through multiple growth factor and cytokine receptors. Science 274, 1194-1197.
75. Sandhu, C., Donovan, J., Bhattacharya, N., Stampfer, M., Worland, P., and Slingerland, J. (2000). Reduction of Cdc25A contributes to cyclin E1-Cdk2 inhibition at senescence in human mammary epithelial cells. Oncogene 19, 5314-5323.
76. Santamaria Babi, L. F., Perez Soler, M. T., Hauser, C., Blaser, K. (1995): Skin-homing T cells in human cutaneous allergic inflammation, Immunol. Res., 14:317-324.
77. Sesto, A., Navarro, M., Burslem, F., Jorcano, J. L. (2002): Analysis of the ultraviolet B response in primary human keratinocytes using oligonucleotide microarrays, Proc. Natl. Acad. Sci. U.S.A., 99:2965-2970.
78. Sexl, V., Diehl, J. A., Sherr, C. J., Ashmun, R., Beach, D., and Roussel, M. F. (1999). A rate limiting function of cdc25A for S phase entry inversely correlates with tyrosine dephosphorylation of Cdk2. Oncogene 18, 573-582.
79. Shibahara, K., Asano, M., Ishida, Y., Aoki, T., Koike, T., Honjo, T. (1995): Isolation of a novel mouse gene MA-3 that is induced upon programmed cell death, Gene, 166:297-301.
80. Shtivelman, E., Sussman, J., and Stokoe, D. (2002). A role for P13-kinase and PKB activity in the G2/M phase of the cell cycle. Curr. Biol. 12, 919-924.
81. Slamon, D. J., Godolphin, W., Jones, L. A., Holt, J. A., Wong, S. G., Keith, D. E., Levin, W. J., Stuart, S. G., Udove, J., and Ullrich, A. (1989). Studies of the HER-2/neu proto-oncogene in human breast and ovarian cancer. Science 244, 707-712.
82. Sluyter, R., Halliday, G. M. (2001): Infiltration by inflammatory cells required for solar-simulated ultraviolet radiation enhancement of skin tumor growth, Cancer Immunol. Immunother., 50:151-156.
83. Stanimirovic, A., Cupic, H., Bosnjak, B., Kruslin, B., Belicza, M. (2003): Expression of p53, bcl-2 and growth hormone receptor in actinic keratosis, hypertrophic type, Arch. Dermatol. Res., 295:102-108.
84. Stern M. C., Gimenez-Conti I. B., Conti C. J. (1995). Genetic susceptibility to papilloma progression in SENCAR mice, Carcinogenesis, 16:1947-1953.
85. Stout, G. J., Westdijk, D., Calkhoven, D. M., Pijper, O., Backendorf, C. M., Willemze, R., Mullenders, L. H., and De Gruijl, F. R. (2005). Epidermal transit of replication-arrested, undifferentiated keratinocytes in UV-exposed XPC mice: an alternative to in situ apoptosis. Proc. Natl. Acad. Sci. U.S. A 102, 18980-18985.
86. Takao, J., Ariizumi, K., Dougherty, 1.1., Cruz, P. D., Jr. (2002): Genomic scale analysis of the human keratinocyte response to broad-band ultraviolet-B irradiation, Photodermatol Photoimmunol. Photomed., 18:5-13.
87. Takeda, K., Kanekura, T., Kanzaki, T. (2004): Negative feedback regulation of phosphatidylinositol 3-kinase/Akt pathway by over-expressed cyclooxygenase-2 in human epidermal cancer cells, J. Dermatol., 31:516-523.
88. Terui, T., Okuyama, R., Tagami, H. (2001): Molecular events occurring behind ultraviolet-induced skin inflammation, Curr. Opin. Allergy Clin. Immunol., 1:461-467.
89. Trempus, C. S., Mahler, J. F., Ananthaswamy, H. N., Loughlin, S. M., French, J. E., and Tennant, R. W. (1998). Photocarcinogenesis and susceptibility to UV radiation in the v-Ha-ras transgenic Tg.AC mouse. J. Invest Dermatol. 111, 445-451.
90. Tripp, C. S., Blomme, E. A., Chinn, K. S., Hardy, M. M., LaCelle, P., Pentland, A. P. (2003): Epidermal COX-2 induction following ultraviolet irradiation: suggested mechanism for the role of COX-2 inhibition in photoprotection, J. Invest. Dermatol., 121:853-861.
91. Tuna, M., Chavez-Reyes, A., and Tari, A. M. (2005). HER2/neu increases the expression of Wilms' Tumor I (W1) protein to stimulate S-phase proliferation and inhibit apoptosis in breast cancer cells. Oncogene 24, 1648-1652.
92. Tyrrell, R. M. (1996). Activation of mammalian gene expression by the UV component of sunlight-1-from models to reality. Bioessays 18, 139-148.
93. Tyrrell, R. M. (1996). UV activation of mammalian stress proteins. EXS 77, 255-271.
94. Vadlamudi, R., Mandal, M., Adam, L., Steinbach, G., Mendelsohn, J., Kumar, R. (1999): Regulation of cyclooxygenase-2 pathway by HER2 receptor, Oncogene, 18:305-314.
95. Vindelov, L. L. (1977). Flow microfluorometric analysis of nuclear DNA in cells from solid tumors and cell suspensions. A new method for rapid isolation and straining of nuclei. Virchows Arch. B Cell Pathol. 24, 227-242.
96. Wan, Y. S., Wang, Z. Q., Shao, Y., Voorhees, J. J., and Fisher, G. J. (2001). Ultraviolet irradiation activates PI 3-kinase/AKT survival pathway via EGF receptors in human skin in vivo. Int. J. Oncol. 18, 461-466.
97. Wang, H. Q., Quan, T., He, T., Franke, T. F., Voorhees, J. J., and Fisher, G. J. (2003). Epidermal growth factor receptor-dependent, NF-kappaB-independent activation of the phosphatidylinositol 3-kinase/Akt pathway inhibits ultraviolet irradiation-induced caspases-3, -8, and -9 in human keratinocytes. J. Biol. Chem. 278, 45737-45745.
98. Wang, N., He, Q., Skog, S., Eriksson, S., Tribukait, B. (2001): Investigation on cell proliferation with a new antibody against thymidine kinase 1, Anal. Cell Pathol., 23:11-19.
99. Way, T. D., Kao, M. C., and Lin, J. K. (2004). Apigenin induces apoptosis through proteasomal degradation of HER2/neu in HER2/neu-overexpressing breast cancer cells via the phosphatidylinositol 3-kinase/Akt-dependent pathway. J. Biol. Chem. 279, 4479-4489.
100. Welss, T., Papoutsaki, M., Michel, G., Reifenberger, J., Chimenti, S., Ruzicka, T., Abts, H. F. (2003): Molecular basis of basal cell carcinoma: analysis of differential gene expression by differential display PCR and expression array, Int. J. Cancer, 104:66-72.
101. White, S. L., Gharbi, S., Bertani, M. F., Chan, H. L., Waterfield, M. D., Timms, J. F. (2004): Cellular responses to ErbB-2 overexpression in human mammary luminal epithelial cells: comparison of mRNA and protein expression, Br. J. Cancer, 90:173-181.
102. Wilson, K. S., Roberts, H., Leek, R., Harris, A. L., Geradts, J. (2002): Differential gene expression patterns in HER2/neu-positive and -negative breast cancer cell lines and tissues, Am. J. Pathol., 161:1171-1185
103. Xia, W., Bisi, J., Strum, J., Liu, L., Carrick, K., Graham, K. M., Treece, A. L., Hardwicke, M. A., Dush, M., Liao, Q., Westlund, R. E., Zhao, S., Bacus, S., and Spector, N. L. (2006). Regulation of survivin by ErbB2 signaling: therapeutic implications for ErbB2-overexpressing breast cancers. Cancer Res. 66, 1640-1647.
104. Xiao, Z., Chen, Z., Gunasekera, A. H., Sowin, T. J., Rosenberg, S. H., Fesik, S., and Zhang, H. (2003). Chk1 mediates S and G2 arrests through Cdc25A degradation in response to DNA-damaging agents. J. Biol. Chem. 278, 21767-21773.
105. Xie, W., Chow, L. T., Paterson, A. J., Chin, E., and Kudlow, J. E. (1999). Conditional expression of the ErbB2 oncogene elicits reversible hyperplasia in stratified epithelia and up-regulation of TGFalpha expression in transgenic mice. Oncogene 18, 3593-3607.
106. Xie, W., Wu, X., Chow, L. T., Chin, E., Paterson, A. J., and Kudlow, J. E. (1998). Targeted expression of activated erbB-2 to the epidermis of transgenic mice elicits striking developmental abnormalities in the epidermis and hair follicles. Cell Growth Differ. 9, 313-325.
107. Yakes, F. M., Chinratanalab, W., Ritter, C. A., King, W., Seelig, S., and Arteaga, C. L. (2002). Herceptin-induced inhibition of phosphatidylinositol-3 kinase and Akt Is required for antibody-mediated effects on p27, cyclin D1, and antitumor action. Cancer Res. 62, 4132-4141.
108. Yu, D. and Hung, M. C. (2000). Role of erbB2 in breast cancer chemosensitivity. Bioessays 22, 673-680.
109. Zhou, B. P., Hung, M. C. (2003): Dysregulation of cellular signaling by HER2/neu in breast cancer, Semin. Oncol., 30:38-48
110. Hung, M. C., Lau, Y. K. (1991) Basic Science of HER-2/neu: A Review, Semin. Oncol. 26(4 Suppl 12):51-9

What is claimed is:

1. A method of suppressing ultraviolet light-induced skin pathologies comprising administration of a therapeutically effective amount of an inhibitor of Erbb2 or HER2 receptor tyrosine kinase activity.

2. The method of claim 1, wherein said administration is topical.

3. The method of claim 1, wherein said administration is prior to UV exposure.

4. The method of claim 1, wherein said administration is concurrent with or after UV exposure.

5. The method of claim 1, wherein said skin pathology is hyperplasia.

6. The method of claim 1, wherein said skin pathology is inflammation.

7. The method of claim 4, wherein said inflammation is sunburn.

8. The method of claim 1, wherein said skin pathology is skin cancer.

9. The method of claim 1, wherein said skin pathology is associated with tumor progression.

10. The method of claim 1, wherein said inhibitor is AG825.

11. A method for reducing adverse effects of UV exposure, wherein the adverse effects are mutagenesis or abnormal proliferation of a cell expressing Erbb2 or HER2, said method comprising administration of a therapeutically effective amount of an inhibitor of Erbb2 or HER2 receptor tyrosine kinase to said cell.

12. A method for inducing apoptosis of a cell expressing Erbb2 or HER2, said method comprising administration of a therapeutically effective amount of an inhibitor of Erbb2 or HER2 receptor tyrosine kinase activity to said cell.

13. A method of enhancing the effect of chemotherapeutic agents that cause S-phase arrest or checkpoint activation, comprising administration of a therapeutically effective amount of an inhibitor of Erbb2 or HER2 receptor tyrosine kinase activity.

14. A line of mouse, wherein the mouse is phenotypically characterized by:
a) lack of Erbb2 expression in cutaneous epithelium;
b) approximately 96% reduction of Erbb2 immunoreactivity in skin cells; and
c) healthy and viable into adulthood and after ultraviolet irradiation.

15. The line of mouse claim 14 whose embryos are deposited with the American Type Culture Collection under accession under ATCC ______.
